(19) United States
(12) Patent Application Publication
(10) Pub. No.: US 2016/0169914 A1
(43) Pub. Date: Jun. 16, 2016

(54) KINETICS BIOMARKERS FOR NEURODEGENERATION

(71) Applicant: KineMed, Inc., Emeryville, CA (US)

(72) Inventors: Marc HELLERSTEIN, Kensington, CA (US); Patrizia FANARA, Pleasanton, CA (US)

(73) Assignee: KineMed, Inc., Emeryville, CA (US)

(21) Appl. No.: 14/857,745

(22) Filed: Sep. 17, 2015

Related U.S. Application Data
(60) Provisional application No. 62/051,884, filed on Sep. 17, 2014.

(51) Int. Cl. G01N 33/68

(52) U.S. Cl. CPC G01N 33/6896; G01N 2458/15; G01N 2800/2835; G01N 2333/4703; G01N 2333/4709; G01N 2800/52

(57) ABSTRACT

The present invention relates to novel diagnostic, prognostic, predictive and pharmacodynamic properties with regard to amyotrophic lateral sclerosis (ALS) and/or Parkinson's disease (PD) with and without dementia components. The methods described herein will prove very useful in the development of diagnostic as well as treatment strategies for patients with amyotrophic lateral sclerosis (ALS) and/or Parkinson's disease (PD).

### CROSS REFERENCE TO RELATED APPLICATIONS

This application claims priority to U.S. Provisional Patent Application Ser. No. 62/051,884, filed Sep. 17, 2014, the disclosure of which is hereby incorporated herein by reference in its entirety for all purposes.

### FIELD OF THE INVENTION

The invention disclosed herein describes novel diagnostic, prognostic, predictive and pharmacodynamic properties with regard to amyotrophic lateral sclerosis (ALS) and Parkinson's disease (PD) with and without cognitive abnormalities such as dementia.

### BACKGROUND OF THE INVENTION

The motoneuron diseases are a group of progressive neurological disorders that damage or destroy both upper (brain) and lower (brain stem and spinal cord) motor neurons, the cells that control voluntary muscle activity such as speaking, walking, breathing, and swallowing. Characteristic symptoms of motoneuron diseases include progressive weakness; loss of strength and loss of muscle mass (wasting); involuntary movements including twitching of muscles; spasticity or stiffness in the arms and legs; and overactive tendon reflexes. Other symptoms of motoneuron diseases can include slowing of voluntary movements (bradykinesias), lack of movement (hypokinesia, masked faces), stereotypical and repeated involuntary movements (choreoathetosis), and frozen postures or restlessness (akathisia). In some types of motoneuron diseases, such as amyotrophic lateral sclerosis (ALS, commonly called Lou Gehrig's disease), muscle weakness is progressive and eventually leads to death, typically associated with loss of respiratory muscle function. Other types of motoneuron diseases progress slowly over the course of many years. Patients with motoneuron disease (MND) are generally free of cognitive impairment, but evidence is growing to support an association between certain subgroups of patients with MND and cognitive abnormalities. For example, motoneuron diseases including ALS and PD can also cause cognitive and mood disturbances. Cognitive impairment in ALS and PD (cortical dementias) is correlated with pathologic and radiographic changes in the cerebral cortex beyond the motor regions (Monastero A. et al. Prevalence and profile of mild cognitive impairment in Parkinson's disease. Neurodegener Dis. 2012; 10(1-4):187-90; Poletti M. et al. Mild cognitive impairment and cognitive-motor relationships in newly diagnosed drug-naive patients with Parkinson's disease. J Neurol Neurosurg Psychiatry 2012; 83(6):601-6; Rippon G A et al. An observational study of cognitive impairment in amyotrophic lateral sclerosis. Arch Neurol. 2006; 63(3):345-52.).

Motoneuron diseases occur in adults and children, and are more common in men than in women. In adults, symptoms usually appear after age 40, and may be non-specific, making diagnosis difficult. In children, particularly in inherited forms of the disease, symptoms may be present from birth. Inherited forms of motoneuron diseases are caused by genetic mutations or deletions that cause degeneration of motor neurons. Nonhereditary motoneuron diseases include ALS (although some hereditary forms do exist) and Parkinson's disease. There are no specific laboratory tests to diagnose Parkinson's disease or ALS with or without dementia.

ALS is an inexorably progressive, invariably fatal disease of the peripheral nervous system. Specifically, ALS is a disease of motor neurons characterized by dysfunction of axons. There is currently no effective treatment. Riluzole (Rilutek®) was approved by the FDA in 1995 but only delays disease progression modestly. In addition to nonhereditary ALS, hereditary forms of ALS exist. Up to 20% of patients with familial ALS have a mutation in the superoxide dismutase (SOD1) gene. This finding allowed the development of a faithful mouse model for ALS. This model, the SOD1-G93A transgenic mouse (“SOD1-G93A TGN mouse”), develops a neurological disorder that mimics ALS and results in death by 18-19 weeks of age.

The SOD1-G93A TGN mouse has become very useful for preclinical discovery and testing of drugs. This particular transgenic mouse model of ALS exhibits higher expression of mutant human Cu, Zn SOD and a shorter course of disease (18-19 weeks). Evaluation of potential therapeutic agents is thereby made faster and more efficient. Also, demonstration of therapeutic benefit in this more aggressive (i.e., high expression) mouse model may provide the most stringent criterion for predicting success in the clinic. Given the expense and time required to organize human clinical trials, only the most active and potent candidate drugs should be brought forward for evaluation in patients. A variety of potential therapeutic agents has been tested in the SOD1-G93A TGN mouse. Other treatment methodologies also have been tested in this model, such as transplantation with human neural stem cells. All treatment modalities tested to date, including Riluzole and neural stem cell transplantation, only delay disease onset and mortality by 20 to 30 days in this model.

The relative lack of success of candidate agents in the SOD1-G93A TGN mouse may reflect the fundamental lack of understanding of the underlying mechanism of motoneuron diseases. More effective diagnostic, prognostic, predictive and pharmacodynamic study methods with regard to understanding motoneuron diseases are needed and the present invention meets these needs.

### SUMMARY OF THE INVENTION

Accordingly, in one aspect the present invention provides a method of diagnosing or predicting the presence of Parkinson's disease (PD) in a patient comprising:
a) examining a plurality of patient samples obtained over one or more timepoints from said patient, wherein said samples comprise isotope-labeled cargo molecules;
b) quantifying the rate of transport of one or more cargo molecules in said samples;
c) comparing the rate of transport of said one or more cargo molecules in said patient samples to the rate of transport of said one or more cargo molecules in control subject samples,
wherein detecting a decrease in the rate of transport of said one or more cargo molecules in said patient samples as compared to the rate of transport of said one or more cargo molecules in said control subject samples is indicative of Parkinson's disease.

In some aspects, the method of diagnosing or predicting the presence of Parkinson's disease (PD) in a patient comprises a step of administering an isotope-labeled substrate to said patient for a period of time sufficient for said isotope label to be detectable in said patient sample, wherein said administration is prior to step a).

In some aspects, the one or more cargo molecules is selected from the group consisting of alpha-synuclein, chromogranin B, chromogranin A, sAPP, and VGF, and combinations thereof.

In some aspects, a decrease in the rate of transport of said one or more cargo molecules selected from the group consisting of chromogranin A, sAPP, and VGF is indicative of longer disease duration.

In some aspects, chromogranin A, sAPP, or VGF do not correlate with disease severity.

In some aspects, a decrease in the rate of transport of said one or more cargo molecules selected from the group consisting of alpha-synuclein and chromogranin B is indicative of degenerating neurons linked to motor symptoms of PD.

In some aspects, the decrease in the rate of transport of said one or more cargo molecules selected from the group consisting of alpha-synuclein and chromogranin B correlates with clinical disease stage, wherein a greater decrease in rate of transport is indicative of a more advanced clinical disease stage and/or disease severity.

In some aspects, said one or more cargo proteins is not neuregulin-1 or clusterin.

In some aspects, said sample is selected from the group consisting of CSF, blood, urine, and tissue. In some aspects, said sample is CSF.

Accordingly, in another aspect, the present invention provides a method of diagnosing or predicting the presence of Parkinson's disease dementia (PDD) in a patient previously diagnosed with Parkinson's disease, the method comprising:
a) examining a plurality of patient samples obtained over one or more timepoints from said patient, wherein said samples comprise isotope labeled cargo molecules;
b) quantifying the rate of transport of one or more cargo molecules in said patient samples;
c) comparing the rate of transport of said one or more cargo molecules in said patient samples to the rate of transport of said one or more cargo molecules in control subject samples, and
d) detecting a decrease in the rate of transport of said one or more cargo molecules in said patient samples as compared to the rate of transport of one or more cargo molecules in said control subject samples,
   wherein the decrease in the rate of transport of said one or more cargo molecules into samples from a subject with PDD as compared to the rate of transport of said one or more cargo molecules into samples from a control subjects is greatly different for a subject with PD as compared to a control subject.

In some aspects, said method comprises a step of administering an isotope-labeled substrate to said patient for a period of time sufficient for said isotope label to be detectable in said patient sample, wherein said administration is prior to step a).

In some aspects, a greater decrease in the rate of transport in said patient samples as compared to said control subject samples results in a greater increase in the probability of developing dementia.

In some aspects, said one or more cargo molecules is selected from the group consisting of proenkephalin-A and galanin.

In some aspects, said sample is selected from the group consisting of CSF, blood, urine, and tissue. In some aspects, said sample is CSF.

Accordingly, in another aspect, the present invention provides a method of differentiating between Parkinson's disease (PD) and amyotrophic lateral sclerosis (ALS) in a patient comprising:
a) examining a plurality of patient samples obtained over one or more timepoints from said patient, wherein said samples comprise isotope labeled cargo molecules;
b) quantifying the rate of transport of one or more cargo molecules in said samples;
c) comparing the rate of transport of said one or more cargo molecules in said patient samples to the rate of transport of said one or more cargo molecules in control subject samples, and
d) detecting a difference in the rate of transport of at least two or more cargo molecules in said patient samples as compared to the rate of transport of said one or more cargo molecules in said control subject samples,
   wherein (i) a decrease in the rate of transport of alpha-synuclein in said patient samples as compared to the rate of transport of alpha-synuclein in said control subject samples and (ii) no change in the rate of transport of neuregulin-1 in said patient samples as compared to the rate of transport of neuregulin-1 in said control subject samples is indicative of PD, and
   wherein (i) a decrease in the rate of transport of neuregulin-1 in said patient samples as compared to the rate of transport of neuregulin-1 in said control subject samples and (ii) no change in the rate of transport of alpha-synuclein in said patient samples as compared to the rate of transport of alpha-synuclein in said control subject samples, is indicative of ALS.

In some aspects, the method comprises a step of administering an isotope-labeled substrate to said patient for a period of time sufficient for said isotope label to be detectable in said patient samples, wherein said administration is prior to step a).

In some aspects, said sample is selected from the group consisting of CSF, blood, urine, and tissue. In some aspects, said sample is CSF.

Accordingly, in another aspect, the present invention provides a method of monitoring a treatment regimen in Parkinson's disease (PD) in a patient being treated for PD with a drug comprising:

a) examining a plurality of patient samples obtained over one or more timepoints from said patient, wherein said samples comprise isotope labeled cargo molecules;
b) quantifying the rate of transport of one or more cargo molecules in said samples;
c) comparing the rate of transport of said one or more cargo molecules in said patient samples to the rate of transport of said one or more cargo molecules in said PD patient samples,

wherein detecting a reduction in the decrease in the rate of transport of said one or more cargo molecules in said patient samples as compared to the rate of transport of one or more cargo molecules in said PD subject samples is indicative of the therapeutic efficacy of the treatment,

wherein said PD subject samples are from (i) a subject not being treated or (ii) from the same patient prior to beginning treatment.

In some aspects, the method comprises a step of administering an isotope-labeled substrate to said patient for a period of time sufficient for said isotope label to be detectable in said patient samples, wherein said administration is prior to step a).

In some aspects, the treatment comprises administration of a drug selected from the group consisting of Azilect (rasagiline) and cholinesterase inhibitors (e.g. donepezil).

In some aspects, the one or more cargo molecules is brain-derived neurotrophic factor (BDNF).

In some aspects, said sample is selected from the group consisting of CSF, blood, urine, and tissue. In some aspects, said sample is CSF.

Accordingly, in another aspect, the present invention provides a method of developing pharmacokinetic treatment profiles for Parkinson's disease (PD) in a patient being treated for PD comprising:
a) examining a plurality of patient samples obtained over one or more timepoints from said patient, wherein said samples comprise isotope labeled cargo molecules;
b) quantifying the rate of transport of at least two or more cargo molecules in said samples;
c) comparing the rate of transport of said at least two or more cargo molecules in said patient samples to the rate of transport of said at least two or more cargo molecules in said PD subject samples,
   wherein detecting a reduction in the decrease in the rate of transport of said one or more cargo molecules in said patient samples as compared to the rate of transport of one more cargo one or more cargo molecules in said PD subject samples is indicative of the therapeutic efficacy of the treatment,
   wherein said PD subject samples are from (i) a subject not being treated or (ii) from the same patient prior to beginning treatment; and
d) developing a signature profile for the differential rates of transport of said at least two or more cargo molecules.

In some aspects, the method comprises a step of administering an isotope-labeled substrate to said patient for a period of time sufficient for said isotope label to be detectable in said patient samples, wherein said administration is prior to step a).

In some aspects, said sample is selected from the group consisting of CSF, blood, urine, and tissue. In some aspects, the sample is CSF.

In some aspects, the treatment comprises administration of rasagiline.

### BRIEF DESCRIPTION OF THE DRAWINGS

FIG. 1 provides data regarding SNCA (alpha synuclein) shown in panel (A) and CHGB (chromogranin B) shown in panel (B). SNCA and CHGB are cargo proteins for which transport rates are biomarkers of motor disability, specifically Parkinson's disease based on Hoehn & Yahr Clinical Test. Correlations of distinct cargo transport rates with Hoehn & Yahr stage in individual PD patients. The data shown in panels (A) and (B) reveal strong correlations between reduced transport rates of alpha-synuclein (SNCA) and chromogranin-B (CHGB) with the Hoehn & Yahr motor disability stage in individual PD patients. Solid lines indicate regression; dashed lines indicate 95% confidence intervals. The statistical test that generated both the r² and P values was a linear regression (n=37 PD patients; mean±SD were generated by technical replicates of repeated sample preparations and analyses). ²H-labeled alpha-synuclein (SNCA) and Chromogranin B (CHGB) are shown in panels (A) and (B), respectively. FIG. 1, panels (C) and (D) show the same markers but include the control, non-PD subjects, showing that quick transport of these two biomarkers (e.g. from one to 3 days, and particularly two days), indicates the patient does not have PD and/or PD symptoms. Taken together, panels (A)-(D) of FIG. 1 shows that the rate of transport of SNCA and CHGB correlate with the Hoehn & Yahr Clinical Test and thus that the measurement of either or both of these markers can be used to diagnose PD and/or determine the severity of the disease. As will be appreciated by those in the art, measuring both markers gives increased confidence in the test, but either biomarker measured alone correlates with PD.

FIG. 2 provides data regarding SNCA (alpha synuclein) shown in panel (A) and CHGB (chromogranin B) shown in panel (B). SNCA and CHGB are cargo proteins for which transport rates are biomarkers of motor disability, specifically Parkinson's disease based on UPDRS Part III Clinical Test. Correlations of distinct cargo transport rates with UPDRS part III scores in individual PD patients. The data shown in panels (A), (B), (C) and (D) of FIG. 2 reveal strong correlations between reduced transport rates of alpha-synuclein (SNCA), shown in panels (A) and (C) and chromogranin-B (CHGB) shown in panels (B) and (D) with the Hoehn & Yahr motor disability scores in individual PD patients. In panels (A) and (B), solid lines indicate regression; dashed lines indicate 95% confidence intervals. The statistical test that generated both the r² and P values was a linear regression (n=37 PD patients; mean±SD were generated by technical replicates of repeated sample preparations and analyses). ²H-labeled SNCA and CHGB are shown in panels (A) and (B), respectively. FIG. 2, panels (C) and (D) show the same markers but include the control, non-PD subjects, showing that quick transport of these two biomarkers (e.g. from one to 3 days, and particularly two days), indicates the patient does not have PD and/or PD symptoms. Taken together, FIG. 2, panels (A)-(D) show that the rate of transport of SNCA and CHGB correlate with the UPDRS Part III score and thus that the measurement of either or both of these markers can be used to diagnose PD and/or determine the severity of the disease. As will be appreciated by those in the art, measuring both markers gives increased confidence in the test, but either biomarker measured alone correlates with PD.

FIG. 3 provides data regarding cargo proteins for which transport rates are biomarkers of disease duration in Parkinson's disease (PD) patients. Panel (A): CHGA (chromogranin A); panel (B): sAPP (soluble amyloid precursor protein) and panel (C): VGF (neurosecretory protein VGF). Correlations of distinct cargo transport rates with disease duration in individual PD patients. Solid lines indicate regression; dashed lines indicate 95% confidence intervals. The statistical test that generated both the r² and P values was a linear regression (n=37 PD patients; mean±SD were generated by technical replicates of repeated sample preparations and analyses). ²H-labeled CHGA, sAPP, and VGF are shown in panels (A), (B), and (C), respectively. FIG. 3, panels (D), (E), and (F) show the same markers but include the control, non-PD subjects, showing that quick transport of these two biomarkers (e.g. from one to 3 days, and particularly two days), indicates the patient does not have PD and/or PD symptoms. (Note that panel (D) shows the sAPP protein while panel (A) shows the full soluble amyloid precursor protein (sAPP)). Taken together, FIG. 3, panels (A)-(F) shows that the rate of transport of SNCA, CHGB and VGF correlate with duration of disease of patients previously diagnosed with PD; and thus that the measurement of any combination or all of these markers can be used to either diagnose PD and/or determine the duration of the disease. As will be appreciated by those in the art, any or all combination of these markers may be used (each alone or sAPP with CHGA, sAPP with VGF, CHGA and VGF, etc.), and that measuring all three markers gives increased confidence in the test, but either biomarker measured alone correlates with PD duration.

FIG. 4 provides data regarding cargo proteins for which transport rates are normal in Parkinson's disease (PD) patients. ²H-labeled Neuregulin-1 (NRG1) and Clusterin (CLU) are shown in panels (A) and (B), respectively.

FIG. 5 provides data regarding that PENK-A (proenkephalin-A), shown in panel (A), and GAL (galanin), shown in panel (B), transport rate differentiates patients with Parkinson's disease (PD) from patients with Parkinson's disease and Dementia (PDD), using MoCA (Montreal Cognition Assessment) Clinical Test. The cargo protein is a biomarker of cognition (based MoCA Scores). Distinct Cargo Transport Rates and Cognitive Impairment in Individual PD Patients. A) Transport rates of proenkephalin-A (PENK-A) and galanin (GAL) measured in CSF of PD patients taking donepezil or rivastigmine for treatment of dementia (filled grey square) and patients that developed cognitive impairment post CSF sampling (filled blue triangle). Open and filled red triangles represent patients with affected and normal cargo transport rates, respectively, and healthy controls are shown by an open black circle. Interestingly, compared to these three PD patients whose MoCA scores indicated cognitive decline and who were on medication for cognitive impairment (e.g. donepezil), alterations in the transport rates of PENK-A and GAL were also reduced in the CSF of a group of patients who were not cognitively impaired on the MoCA (n=12 patients represented with filled blue and open red triangles). However, clinical follow-up revealed that 3 of the 12 patients with slower transport rates for PENK-A and GAL (displayed with a filled blue triangle) indeed developed clinical symptoms and were prescribed cholinesterase inhibitors (e.g. donepezil) for cognitive impairment within 3 years of the CSF measurements.

FIG. 6 provides data regarding that galanin transport rate differentiates patients with Parkinson's disease (PD) from patients with Parkinson's disease and Dementia (PDD), using MOCA scores. Panel (A) shows PDD and PD patient data; and panel (B) shows Controls.

FIG. 7 provides data regarding that BDNF (brain-derived neurotrophic factor) transport rate is a biomarker of response to Azilect (rasagiline) therapy in Parkinson's disease (PD) and that there was significant improvement of BNDF transport rates in PD patients treated with Azilect (rasagiline). Additionally, there was significant improvement in transport rates of BDNF in CSF of PD patients treated with Azilect. Panel (A): PD patients taking rasagiline 1 mg/day showed reduced deficits in transport rates of BDNF, as compared to patients not treated with Azilect. Statistical significance was assessed by 1-way ANOVA with Tukey post-hoc testing. A P value less than 0.05 was considered statistically significant (* P<0.05). Panel (B): A strong correlation was observed between reduced delays in the rates of BDNF transport and the duration of treatment with Azilect (years prior to LP). Solid lines indicate regression; dashed lines indicate 95% confidence intervals. The statistical test that generated both the r² and P values was a linear regression (mean±SD were generated by technical replicates of repeated sample preparations and analyses). Taken together, FIG. 7, panels (A) and (B) show that not only can BDNF be used as a marker for the diagnosis of PD, it can also be used as a measure of drug efficacy in PD.

FIG. 8 provides data regarding neuregulin-1 (NRG1) and alpha-synuclein (SNCA) transport rates which distinguish ALS (amyotrophic lateral sclerosis; Lou Gehrig's Disease) from Parkinson's disease (PD). Neuronal subpopulation-specific cargo data is provided. FIG. 8 thus shows the remarkable ability to distinguish between PD and ALS patients using two markers, NRG1 and SNCA.

FIG. 9 Chromogranin B (CHGB) and Chromogranin A (CHGA) transport rates are altered in ALS (amyotrophic lateral sclerosis; Lou Gehrig's Disease) and PD as compared to healthy controls.

FIG. 10 depicts the use of a number of biomarkers that distinguish PD patients from healthy controls, the use of any one or more, in any combination, finds use in the present invention for diagnosing and/or evaluating the status of PD patient. The data describes a cross-sectional study to evaluate the correlation between delays in axonal transport rates and disease severity. Patients are categorized by the number of years since diagnosis as early stage (within 4 years of diagnosis), moderately advanced (within 5-7 years of diagnosis) and severe (8 or more years of diagnosis). Differential delays in transport rates of neuronal cargo proteins in CSF of PD subjects were measured by a single lumbar puncture. Distinct transport times for 10 CSF cargo proteins, at the time of sampling, are shown in CSF of the PD patients (N=37) compared to non-PD healthy controls (N=13). Mean±SD were generated by technical replicates of repeated sample preparations and analyses.

FIG. 11 depicts different transport rates from two Parkinson's disease patients. CSF samples were collected from PD subjects at two different times by two consecutive lumbar punctures (LPs) to detect changes in transport rates over time. The decrease in the rate of transport of alpha-synuclein (SNCA) is graphed as the “Rate of Neurodegeneration” over time. This is obtained by calculating the difference in transport rates of SNCA over time (e.g. SNCA transport rate in these 2 PD subjects minus the mean of SNCA transport rate in healthy controls divided by the time between the two LPs). UPDRS part III motor disability scores in these PD patients at the two time points of CSF sampling (LPs) were: Patient #1 (LP1 UPDRS part III=11; LP2 UPDRS part III=19) Patient #2 (LP1 UPDRS part III=15; UPDRS part III=20). Mean±SD were generated by technical replicates of repeated sample preparations and analyses.

FIG. 12 depicts isotopic enrichment of 3 representative peptides synthesized at various levels of body water enrichment. Relationships can be described by a quadratic fit through the origin.

FIG. 13 provides a schematic overview of CSF fractionation from humans and animal models for kinetic measurements of cargo transport rates.

### DETAILED DESCRIPTION OF THE INVENTION

### Overview

The present invention describes novel diagnostic, prognostic, predictive and pharmacodynamic properties with regard to amyotrophic lateral sclerosis (ALS) and Parkinson's disease (PD) with and without dementia (Parkinson' disease dementia, PDD) components. Such methods are very useful in the development of diagnostic, prognostic, and treatment strategies for patients with either ALS or PD.

“Cargo” molecules travel from the cell body of a motor neuron, their primary site of production, to the nerve terminal where they are released into the synaptic cleft. The present disclosure is directed to the discovery that the transport rates of cargo molecules, generally down the axon of neurons that then are released from the cell into patient samples, such as CSF, are markedly different in different motorneuronal diseases such as amyotrophic lateral sclerosis (ALS) and Parkinson's disease (PD). These biomarkers can be quantitated and their rate of transport measured, such that better diagnosis and disease prediction, as well as prognosis, can occur. These analyses can also lead to better treatment regimen development as well as allowing for patients to be prescribed proper treatments. The present methods can also be used by physicians to monitor treatments and change treatment regimens by monitoring transport rates of cargo molecules as described herein. Additionally, analysis of the rates of transport can also be employed to predict dementia and/or to predict development of dementia. Additionally, the methods described herein can be used to generate pharmacokinetic profiles with regard to rates of transport of cargo molecules into patient samples, such as CSF, which can be used to assist with patient care. Finally, the present invention provides for method of monitoring and/or screening the effect and efficacy of drugs (including drug candidates) in a subjective way. For example, patients can be tested, treated with drugs and retested, to see if the rates of transport improve.

These methods will dramatically improve the tools available to enhance the care available for amyotrophic lateral sclerosis (ALS) and Parkinson's disease (PD), and hopefully lead to a better quality of life for such individuals.

The highly asymmetric morphology of neurons, characterized by the presence of axodendritic processes that can reach in length several orders of magnitude the diameter of the cell body, is determined by the capacity of the cytoskeleton to sustain such processes and to support the transport of organelles, vesicles, or protein subunits and complexes (“cargo molecules”) over very long distances. One of the major cytoskeletal systems is the microtubule-based transport system along which kinesin and dynein motor proteins generate force and drive the traffic of many cellular components (e.g. synaptic vesicles). Microtubule (MT)-mediated axonal transport is the main neuronal communication and transportation network, which serves as the pathway for secreted neurotransmitters of various types, including growth factors, neuropeptides, enzymes, and glycoproteins. While not wanting to be bound by theory, it is generally understood that “cargo molecules” travel from the cell body of a motor neuron, their primary site of production, to the nerve terminal inside the synaptic vesicles (SVs) to be released into the axonal synaptic cleft via axonal transport. The cerebrospinal fluid (CSF) is a medium for the release cargo molecules and the cargo molecules may also circulate in the blood or urine, as well as other biological samples.

As used herein, the phrases “cargo molecule” or “cargo molecules” include any molecules transported by the synaptic vesicles via transport, while not being bound by theory this is generally transport down a neuron, referred to as “axonal transport”, followed by release from the neuron. In some embodiments, these molecules are referred to as “brain-derived cargo molecules”, indicating that the molecules are derived directly from neurons (brain cells). These cargo molecules can then be sampled in patient samples, such as CSF. Such cargo molecules include but are not limited to proteins, protein subunits, molecular complexes, or organelles. Cargo molecules of the present disclosure include but are not limited to chromogranin B, chromogranin A, proenkephalin A, neurosecretory protein VGF, clusterin, alpha-synuclein, soluble amyloid precursor protein (sAPP), brain-derived neurotrophic factor (BDNF), galanin, and neuregulin-1.

Chromogranin B (CHGB), also known as secretoneurin or secretogranin I, is a 33-amino acid neuropeptide derived from secretogranin II (chromogranin C). It is a tyrosine-sulfated secretory protein found in a wide variety of peptidergic endocrine cells and functions as a neuroendocrine secretory granule protein which may be the precursor for other biologically active peptides.

Chromogranin A (CHGA) is a 48 kDa protein member of the granin family of neuroendocrine secretory proteins. Chromogranin A is a 439 amino-acid protein generated from a 457 amino acid pre-chromogranin A protein. Chromogranin A is a soluble protein co-stored and co-released along with resident catecholamines and polypeptide hormones or cell-specific neurotransmitters. It is located in secretory vesicles of neurons and endocrine cells such as islet beta cell secretory granules in pancreas.

Proenkephalin A is an endogenous opioid polypeptide hormone which, via proteolyic cleavage, produces the enkephalin peptides. It plays a role in a number of physiologic functions, including regulation of synaptic plasticity, pain perception and responses to stress. Furthermore it is cleaved into synenkephalin, met-enkephalin, PENK (114-133), PENK (143-183), met-enkephalin-Arg-Gly-Leu, Leu-enkephalin, PENK (237-258), and met-enkephalin-Arg-Phe.

Neurosecretory protein VGF may be involved in the regulation of cell-cell interactions or in synatogenesis during the maturation of the nervous system. It is generally found in the central and peripheral nervous systems and is synthesized exclusively in neuronal and neuroendocrine cells. Furthermore it is cleaved into neuroendocrine regulatory peptide-1 and neuroendocrine regulatory peptide-2.

Clusterin is a 75-80 kDa disulfide-linked heterodimeric protein associated with the clearance of cellular debris and apoptosis. It is expressed in most mammalian tissues and can be found in blood plasma, milk, urine, cerebrospinal fluid and semen. Clusterin can be cleaved into clusterin beta chain and clusterin alpha chain.

Amyloid precursor protein (APP) is the precursor molecule which undergoes proteolysis to generate soluble precursor proteins (sAPP). sAPP is involved in a variety of neuronal effects, including communication and proliferation effects.

Brain-derived neurotrophic factor (BDNF) is 119 amino acid protein which is a member of the neurotrophin family of growth factors and binds to at least two receptors, TrkB and the LNGFR (low-affinity nerve growth factor receptor, also known as p75). BDNF acts on neurons of the central nervous system and the peripheral nervous system and generally functions to support the survival of existing neurons as well as encourage the growth and differentiation of new neurons and synapses.

Alpha-synuclein (also referred to as SNCA) is a protein is made of 140 amino acids and is encoded by the SNCA gene. alpha-synuclein is found in presynaptic terminals where the protein interacts with phospholipids other proteins in the presynaptic terminals. Alpha-synuclein may also be involved in maintaining the supply of synaptic vesicles at presynaptic terminals as well as regulating the release of neurotransmitters.

Galanin is a neuropeptide widely expressed in the brain, spinal cord, and gut of humans as well as other mammals. Galanin is predominately involved in the modulation and inhibition of action potentials in neurons. Galanin is a 29 amino-acid peptide (30 amino-acids in humans) and is produced from the cleavage of a 123 amino-acid protein known as preprogalanin.

Neuregulin-1 (NRG-1) extracellularly is a protein made of 242 amino acids and is encoded by the NRG1 gene. Neuregulin-1 is expressed predominantly in neural tissue, respiratory epithelium, and endocardiumis. Neuregulin-1 plays important roles in the development and plasticity of the brain and mediates cell-cell interactions and survival.

Microtubules (“MTs”) are abundant in neurons, where they facilitate the formation of, and confer stability to, neurites (axons and dendrites). They are the primary determinant of neuronal morphology and facilitate the formation of, and confer stability to, neurites (axons and dendrites). The process of assembly and disassembly of axonal microtubules (known as “microtubule dynamics”) underlies their ability to determine and maintain neuronal morphology. This process, essential for the structural stability of the neuron, also represents a signaling pathway within neurons. Microtubule dynamics is regulated largely by microtubule-associated proteins (MAPs). The neuronal MAPs have a specific polar distribution and play a prominent role in the stabilization of microtubules.

Neurons such as motor neurons have several distinct populations of neuronal microtubules, generally classified by the MAPs to which they bind. By “neuronal microtubules” is meant a protein structure composed of polymers of tubulin, occurring singly, in pairs, triplets or bundles in living cells. By “tubulin” is meant the principal protein component of microtubules. Tubulin is a dimer composed of two globular polypeptides, alpha-tubulin and beta-tubulin (α-tubulin and β-tubulin). Microtubules are assembled from dimers α-tubulin and β-tubulin.

Neuronal microtubules are present in different neuronal compartments (e.g., soma, dendrites and axons) and in association with different MAPs (e.g., tau, MAP2 and STOP). Microtubules are required to establish and maintain neuronal differentiation and long-distance transport of neurotransmitter substances along the axons to distant synapses.

In general, there are three main classes of neuronal microtubules: growth cone (also known as “axonal distal” or “axonal tip”) microtubules (also referred to herein and in the figures as “tau-MTs”); dendritic microtubules (also referred to herein as “MAP-2 MTs”), and hillock and axonal shaft microtubules (also referred to herein and in the figures as “STOP-MTs”). In general, the terminology arises from the microtubule-associated proteins that bind each category. “MAPs” or “microtubule-associated proteins” are proteins that, upon binding to a microtubule, alter its function and/or behavior. Thus, for example, capture of growth cone and axonal distal microtubules is done by using affinity binding to tau antibody. The tau-unbound material (the dendritic microtubules) is then captured by affinity binding to MAP2 antibody, leaving only hillock and axonal shaft microtubule (STOP-MTs) in the MAP2-unbound fraction. Alternatively, STOP-MTs can be directly isolated by exploiting their unique ability, compared to other MT subpopulations, to resist depolymerization in cold temperatures and millimolar concentration of CaCl₂.

As used herein, “tau” or “tau protein” or “tau MAP” is a major class of microtubule-associated proteins (MAPs) isolated from the brain. In nerve cells tau is highly enriched in the axonal growth cone. Tau proteins promote the nucleation (initiation) process of tubulin polymerization in vitro. Tau is known to be a regulator of the turnover/assembly of dynamic axonal growth cone microtubules in the brain. Chemically modified tau proteins also appear to be involved in the formation and/or composition of the neurofibrillary tangles and neuropil threads found in Alzheimer's disease.

As used herein, “MAP2” or “Microtubule-Associated Protein-2” is a high molecular weight microtubule-associated protein that is highly enriched in neuronal dendritic microtubules. Under certain conditions, MAP2 is required for tubulin assembly into microtubules and stabilizes the assembled microtubules, regulating their dynamics.

As used herein, “STOP” or “Stable Tubule Only Polypeptide” is a neuronal Ca²⁺-calmodulin-regulated microtubule associated protein. STOP stabilizes microtubules indefinitely against in vitro disassembly induced by cold temperature, millimolar calcium or drugs.

By “neuronal cold-stable microtubules” is meant an abundant subpopulation of axonal microtubules that are stable to disassembly induced by both drugs and cold-temperature. Resistance to microtubule disassembly by drugs and cold-temperature is largely due to polymer association with STOP.

The term “individual” refers to a vertebrate, usually a mammal, particularly a human. A “patient” is a human subject, either a control patient, e.g. one without the disease being evaluated, or a patient either previously diagnosed with the disease or suspected of having a disease.

The term “mammal” is meant any member of the class Mammalia including but not limited to, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

In general, the term “test subject” or “test patient” as used herein is an individual who is being evaluated with regard to a motoneuron disease. The individuals may be an individual who has or has not yet developed symptoms, is at early clinical stages of the diseases, is a late stages of the disease or is at any stage of the disease but has not yet developed dementia.

The terms “sample” or “biological sample” as used herein refer to any of a variety of biological samples can be taken in any test living systems (i.e., patients or subjects, including control and non-control patients or subjects) and any biological fluid samples can find use with the methods of the present disclosure. Any biological sample which contains cargo molecules can find use with the methods of the present disclosure. Examples of motoneuron samples include but are not limited to, sciatic or peripheral nerve tissue and samples from the motor cortex of the brain sampling cerebrospinal fluid (CSF). Other samples that find use with the present methods include blood, urine or tissue samples.

The term “treatment” in the instant invention is meant to include therapeutic treatment, as well as prophylactic, or suppressive measures for the disease or disorder.

Those “in need of treatment” include mammals already having the disease or disorder, including those whose symptoms are to be delayed in onset or ameliorated, as well as those prone to having the disease or disorder, including those in which the disease or disorder is to be prevented.

“Therapeutic efficacy” can be measured based on a variety of factors, including an observed cargo molecule rate of transport that is closer to or more similar to a control rate of transport. Therapeutic efficacy can also include alleviation of any or all disease symptoms.

### Parkinson's Disease: Methods for Diagnosis and Prognosis

In some embodiments, the present disclosure provides methods for diagnosing and/or predicting the presence of Parkinson's disease (PD) in a patient. The method includes the steps of a) examining a plurality of patient samples obtained over one or more timepoints from said patient, said samples comprise isotope labeled cargo molecules; b) quantifying the rate of transport of one or more cargo molecules into said samples or in said samples; c) comparing the rate of transport of said one or more cargo molecules into said patient samples or in said patient samples to the rate of transport of said one or more cargo molecules control subject samples, wherein detecting a decrease in the rate of transport of said one or more cargo molecules into said patient samples or in said patient samples as compared to the rate of transport of one or more cargo molecules into said control subject samples or in said control subject samples is indicative of Parkinson's disease.

In some embodiments, a plurality of samples includes one or more samples.

Generally, the method further comprises a step of administering an isotope-labeled substrate to said patient for a period of time sufficient for said isotope-labeled substrate or isotope label to be detectable in said patient samples, wherein administration is prior to step a) of the method. Such methods are described in detail herein below. In general, as more fully described below, patients drink isotope-labeled water (e.g., heavy water, D₂O or ²H₂O) over a period of days, generally seven, during which time the substrate is synthesized (i.e., incorporated) into the proteins of the body, including cargo molecules. By sampling at a later time point (generally several weeks later, from 14 to 21 days) and calculating the ratio of isotope-labeled cargo molecules (biomarkers) as a fraction of total cargo molecules, the rate of transport can be calculated and then compared to control subjects. In some embodiments, the timepoint is taken at 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, or more days post isotope-labeled substrate. In some embodiments, the timepoint is taken at 14 days, 15 days, 16 days, 17 days, 18 days, 19 days, 20 days, or 21 days post isotope-labeled substrate.

The rate of transport can be measured by employing the isotope measurement methods known in the art or those described in detail herein below. A decrease in the rate of transport can also be referred to as an increase in the time necessary for one or more cargo molecules to be transported into a patient sample, such as CSF. In some embodiments, the decrease is a decrease in the patient rate of transport as compared to the control rate of transport of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

In some embodiments, a decrease in the rate of transport is referred to as an increase in the amount of time necessary for one or more cargo molecules to be detectable in said patient sample, such as CSF. In some embodiments, the decrease in the rate of transport is an increase in the amount of time necessary for one or more cargo molecules to be detectable in a patient sample as compared to the amount of time necessary for one or more cargo molecules to appear and be detectable in a control subject sample of at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, or at least about a month.

In some embodiments, the one or more cargo molecules whose transport rate is detected in a patient sample in order to diagnose or predict PD in a patient include alpha-synuclein, chromogranin B, chromogranin A, sAPP, and VGF, and combinations thereof. In some embodiments, the rate of transport is quantified for 1, 2, 3, 4, or 5 of the recited cargo molecules. In some embodiments, the rate of transport is quantified for alpha-synuclein. In some embodiments, the rate of transport is quantified for chromogranin B. In some embodiments, the rate of transport is quantified for chromogranin A. In some embodiments, the rate of transport is quantified for sAPP. In some embodiments, the rate of transport is quantified for VGF. In some embodiments, the rate of transport is quantified for a combination of alpha-synuclein and chromogranin B. In some embodiments, the rate of transport is quantified for a combination of chromogranin A, sAPP and VGF.

In some embodiments, the one or more cargo proteins are not neuregulin-1 and/or clusterin.

In some embodiments the decrease in the rate of transport of said one or more cargo molecules is indicative of longer duration of PD (i.e., that the patient has been diagnosed with PD for a longer amount of time). In some embodiments disease duration is measure in years. In some embodiments, the duration of PD is about 1 year, about 5 years, about 10 years, about 15 years, or about 20 years. In some embodiments, a decrease in the rate of transport of chromogranin A, sAPP, and/or VGF into a patient sample is indicative of longer disease duration (i.e., that the patient has been diagnosed with PD for a longer amount of time).

While a decrease in the rate of transport of chromogranin A, sAPP, and/or VGF into a patient sample can be indicative of longer duration of PD, the decrease in transport rate for chromogranin A, sAPP, and/or VGF does not correlate with disease severity (i.e., the severity of the presentation of the symptoms of PD).

In some embodiments, a decrease in the rate of transport of alpha-synuclein and/or chromogranin B from a patient as compared to the rate of transport from a control subject is indicative of degenerating neurons linked to or associated with motor symptoms of PD—

In some embodiments, the decrease in the rate of transport of alpha-synuclein and/or chromogranin B from a patient as compared to the rate of transport from a control subject correlates with clinical disease stage. A greater or more significant decrease in rate of transport is indicative of a more advanced clinical disease stage and/or disease severity. In some embodiments, the decrease in the rate of transport correlates with a Hoehn & Yahr scale score (Hoehn, M. and Yahr M., “Parkinsonism: onset, progression and mortality” Neurology, 17 (5): 427-442 (1967)). The Hoehn & Yarn scale score is a commonly used system for describing the progress of Parkinson's disease symptoms that was published in 1967 and included stages 1 through 5. Since then, the scale has been modified with the addition of stages 1.5 and 2.5 to help describe the intermediate course of the disease. A summary of the Hoehn & Yarn scale is provided in Table 1 below.

TABLE 1
Hoehn & Yarn Scale Summary

| | | Modified Hoehn and Yahr |
|---|---|---|
| Stage | Hoehn and Yahr Scale | Scale |
| 1 | Unilateral involvement only | Unilateral involvement |
| | usually with minimal or no | only |
| | functional disability | |
| 1.5 | — | Unilateral and axial |
| | | involvement |
| 2 | Bilateral or midline involvement | Bilateral involvement without |
| | without impairment of balance | impairment of balance |
| 2.5 | — | Mild bilateral disease with |
| | | recovery on pull test |
| 3 | Bilateral disease: mild to | Mild to moderate bilateral |
| | moderate disability with | disease; some postural |
| | impaired postural reflexes; | instability; physically |
| | physically independent | independent |
| 4 | Severely disabling disease; | Severe disability; still able |
| | still able to walk or stand | to walk or stand unassisted |
| | unassisted | |
| 5 | Confinement to bed or wheelchair | Wheelchair bound or |
| | unless aided | bedridden unless aided |

In some embodiments, a decrease in the patient rate of transport as compared to the control rate of transport of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% correlates to a Hoehn & Yarn scale score of 1, 1.5, 2, 2.5, 3, 4, or 5.

In some embodiments, a decrease in the rate of transport is referred to as an increase in the amount of time necessary for one or more cargo molecules to appear and to be detectable in a patient sample. In some embodiments, the decrease in the rate of transport in a patient as compared to the rate of transport in a control subject by at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, or at least about a month correlates to a Hoehn & Yarn scale score of 1, 1.5, 2, 2.5, 3, 4, or 5.

In some embodiments, a patient rate of transport of about 4 days to about 6 days, or about 4 days, about 5 days, or about 6 days correlates to a Hoehn & Yarn scale score of 1. In some embodiments, a patient rate of transport of about 5 days, or about 6 days correlates to a Hoehn & Yarn scale score of 1.5. In some embodiments, a patient rate of transport of about 5 days to about 12 days, about 7 days to about 12 days, or about 7 days, about 8 days, about 9 days, about 10 days, about 11 days or about 12 days correlates to a Hoehn & Yarn scale score of 2. In some embodiments, a patient rate of transport of about 8 days to about 13 days, or about 8 days, about 9 days, about 10 days, about 11 days, about 12, or about 13 days correlates to a Hoehn & Yarn scale score of 2.5. In some embodiments, a patient rate of transport of about 8 days to about 14 days, or about 8 days, about 9 days, about 10 days, about 11 days, about 12 days, about 13 days, or about 14 days correlates to a Hoehn & Yarn scale score of 3. In some embodiments, the cargo molecule whose rate of transport is decreased and which correlates with the Hoehn & Yarn scale score is alpha-synuclein.

In some embodiments, a patient rate of transport of about 4 days to about 5 days, or about 4 days, about 5 days correlates to a Hoehn & Yarn scale score of 1. In some embodiments, a patient rate of transport of about 5 days correlates to a Hoehn & Yarn scale score of 1.5. In some embodiments, a patient rate of transport of about 4 days to about 7 days, or about 5 days to about 7 days, or about 4 days, about 5 days, about 6 days or about 7 days correlates to a Hoehn & Yarn scale score of 2. In some embodiments, a patient rate of transport of about 9 days to about 9 days, or about 6 days, about 7 days, about 8 days, or about 9 days correlates to a Hoehn & Yarn scale score of 2.5. In some embodiments, a patient rate of transport of about 7 days to about 11 days, or about 7 days, about 8 days, about 9 days, about 10 days, or about 11 days correlates to a Hoehn & Yarn scale score of 3. In some embodiments, the cargo molecule whose rate of transport is decreased and which correlates with the Hoehn & Yarn scale score is chromogranin B.

In some embodiments, the decrease in the rate of transport of alpha-synuclein and/or chromogranin B from a patient as compared to the rate of transport from in a control subject correlates with clinical disease stage. A greater or more significant decrease in rate of transport is indicative of a more advanced clinical disease stage and/or disease severity. In some embodiments, the decrease in the rate of transport correlates with a UPDRS Part III score. The unified Parkinson's disease rating scale (UPDRS) is used to follow the longitudinal course of Parkinson's disease. The UPDRS rating scale is another commonly used scale in the clinical study of Parkinson's disease. (Ramaker, C., et al., “Systematic evaluation of rating scales for impairment and disability in Parkinson's disease”, Movement Disorders, 17 (5): 867-876 (2002)). The UPDRS scale is titled; (1) nonmotor experiences of daily living (13 items), (2) motor experiences of daily living (13 items), (3) motor examination (18 items), and (4) motor complications (six items) and each subscale now has 0-4 ratings, where 0=normal, 1=slight, 2=mild, 3=moderate, and 4=severe. A summary of the UPDRS scale is provided in Table 2 below.

TABLE 2
UPDRS Scale Summary

| Part | Title |
|---|---|
| Part I | Evaluation of mentation, behavior, and |
| | mood |
| Part II | Self-evaluation of the activities of daily life |
| | (ADLs) including speech, swallowing, |
| | handwriting, dressing, hygiene, falling, |
| | salivating, turning in bed, walking, and |
| | cutting food |
| Part III | Clinician-scored monitored motor |
| | evaluation |
| Part IV | Complications of therapy |
| Part V | Hoehn and Yahr staging of severity of |
| | Parkinson's disease |
| Part VI | Schwab and England ADL scale |

In some embodiments, a decrease in the patient rate of transport as compared to the control rate of transport of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% correlates to a UPDRS Part III score of about 10, about 15, about 20, about 25, about 30, or about 32.

In Some embodiments, a decrease in the rate of transport is referred to as an increase in the amount of time necessary for one or more cargo molecules to appear and to be detectable in a patient sample. In some embodiments, the decrease in the rate of transport in a patient as compared to the rate of transport in a control subject by at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days correlates to a UPDRS Part III score of about 10, about 15, about 20, about 25, about 30, or about 32.

In some embodiments, a patient rate of transport of about 4 days to about 14 days, correlates to a UPDRS Part III score of 10 to 33. In some embodiments, a patient rate of transport of about 4 days correlates to a UPDRS Part III score of about 10. In some embodiments, a patient rate of transport of about 7 days correlates to a UPDRS Part III score of about 15. In some embodiments, a patient rate of transport of about 9 days correlates to a UPDRS Part III score of about 20. In some embodiments, a patient rate of transport of about 11 days correlates to a UPDRS Part III score of about 25. In some embodiments, a patient rate of transport of about 4 days correlates to a UPDRS Part III score of about 30. In some embodiments, a patient rate of transport of about 14 days correlates to a UPDRS Part III score of about 32. In some embodiments, the cargo molecule whose rate of transport is decreased and which correlates with the UPDRS Part III score is alpha-synuclein.

In some embodiments, a patient rate of transport of about 4 days to about 10 days, correlates to a UPDRS Part III score of 10 to 33. In some embodiments, a patient rate of transport of about 4 days correlates to a UPDRS Part III score of about 10. In some embodiments, a patient rate of transport of about 5 days correlates to a UPDRS Part III score of about 15. In some embodiments, a patient rate of transport of about 6 days correlates to a UPDRS Part III score of about 20. In some embodiments, a patient rate of transport of about 8 days correlates to a UPDRS Part III score of about 25. In some embodiments, a patient rate of transport of about 9 days correlates to a UPDRS Part III score of about 30. In some embodiments, a patient rate of transport of about 10 days correlates to a UPDRS Part III score of about 32. In some embodiments, the cargo molecule whose rate of transport is decreased and which correlates with the UPDRS Part III score is chromogranin B.

### Parkinson's Disease: Methods for Diagnosis and Prediction of Dementia

In some embodiments, the present disclosure provides methods for diagnosing or predicting the presence of Parkinson's disease dementia (PDD) in a patient previously diagnosed with Parkinson's disease. The method includes the steps of a) examining a plurality of patient samples obtained over one or more timepoints from said patient, wherein said samples comprise isotope labeled cargo molecules; b) quantifying the rate of transport of one or more cargo molecules into said samples; c) comparing the rate of transport of said one or more cargo molecules into said samples or in said samples to the rate of transport of said one or more cargo molecules into said control subject samples or in said control subject samples, and d) detecting a decrease in the rate of transport of said one or more cargo molecules into said patient samples or in said patient samples as compared to the rate of transport of one or more cargo molecules into said control subject samples or in said control subject samples, wherein the decrease in the rate of transport for samples from a subject with PDD as compared to the rate of transport in samples from a control subjects is greater than the decrease in the rate of transport for a subject with PD as compared to the rate of transport in samples from a control subject.

In some embodiments, a plurality of samples includes one or more samples.

In some embodiments, the method further comprises a step of administering an isotope-labeled substrate to said patient for a period of time sufficient for said isotope-labeled substrate or isotope label to be detectable in said patient samples, wherein administration is prior to step a) of the method. Such methods are described in detail herein below. In general, as more fully described below, patients drink isotope-labeled water (e.g., heavy water, D₂O or ²H₂O) over a period of days, generally seven, during which time the substrate is synthesized (i.e., incorporated) into the proteins of the body, including cargo molecules. By sampling at a later time point (generally several weeks later, from 14 to 21 days) and calculating the ratio of isotope-labeled cargo molecules (biomarkers) as a fraction of total cargo molecules, the rate of transport can be calculated and then compared to control subjects. In some embodiments, the timepoint is taken at 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, or more days post isotope-labeled substrate. In some embodiments, the timepoint is taken at 14 days, 15 days, 16 days, 17 days, 18 days, 19 days, 20 days, or 21 days post isotope-labeled substrate.

The rate of transport can be measured by employing the isotope measurement methods known in the art or those described in detail herein below. A decrease in the rate of transport can also be referred to as an increase in the time necessary for the one or more cargo molecules to be transported into a patient sample. In some embodiments, the decrease is a decrease in the patient rate of transport as compared to the control rate of transport of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

In some embodiments, a decrease in the rate of transport is referred to as an increase in the amount of time necessary for one or more cargo molecules to appear and to be detectable in a patient sample. In some embodiments, the decrease in the rate of transport is an increase in the amount of time necessary for one or more cargo molecules to appear and to be detectable in a sample from a patient as compared to the amount of time necessary for one or more cargo molecules to appear and to be detectable in a sample from a control subject of at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, or at least about a month.

Not only is a decrease in the rate of transport higher or increased when diagnosing or predicting dementia in PD patients (PDD), but also important is the magnitude of the decrease in the rate of transport in PD patients. A greater or larger decrease (i.e., a greater deviation or change in the patient rate of transport from the control subject rate of transport) correlates with a greater increase in the probability a PD patient has of developing dementia (PDD). In some embodiments, a greater decrease in the rate of transport in said patient samples as compared to said control subject samples results in a greater increase in the probability of developing dementia. In some embodiments, the decrease in the rate of transport is an increase in the amount of time necessary for one or more cargo molecules to appear and to be detectable in a sample from a PDD subject as compared to the amount of time necessary for one or more cargo molecules to appear and to be detectable in a sample from a control subject of at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, or at least about a month.

In some embodiments, a greater decrease in the rate of transport in said patient samples as compared to said control subject samples is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% greater.

In some embodiments, the one or more cargo molecules whose transport rate is detected in order to diagnose dementia in a PD patient or predict dementia in a PD patient include proenkephalin-A and galanin, and combinations thereof. In some embodiments, the rate of transport is quantified for 1 or 2 of the recited cargo molecules. In some embodiments, the rate of transport is quantified for proenkephalin-A. In some embodiments, the rate of transport is quantified for galanin. In some embodiments, the rate of transport is quantified for proenkephalin-A and galanin.

In some embodiments, the decrease in the rate of transport of proenkephalin-A and/or galanin from a patient as compared to the rate of transport from a control subject correlates with dementia onset or probability of developing dementia. A greater or more significant decrease in the rate of transport is indicative of an increased probability of developing dementia. In some embodiments, the decrease in the rate of transport correlates with a MoCA score. The Montreal Cognitive Assessment (MoCA) was created in 1996 has been validated and used to evaluate cognitive abilities in clinical settings (Nasreddine Z. S., et al., “The Montreal Cognitive Assessment (MoCA): A Brief Screening Tool For Mild Cognitive Impairment”, Journal of the American Geriatrics Society 53:695-699 (2005)).

The MoCA assesses multiple cognitive domains. The short-term memory recall task (5 points) involves two learning trials of five nouns and delayed recall after approximately 5 minutes. Visuospatial abilities are assessed using a clock-drawing task (3 points) and a three-dimensional cube copy (1 point). Multiple aspects of executive functions are assessed using an alternation task adapted from the trail-making B task (1 point), a phonemic fluency task (1 point), and a two-item verbal abstraction task (2 points). Attention, concentration and working memory are evaluated using a sustained attention task such as target detection using tapping (1 point), a serial subtraction task (3 points), and digits forward and backward (1 point each). Language is assessed using a three-item confrontation naming task with low-familiarity animals (lion, camel, rhinoceros; 3 points), repetition of two syntactically complex sentences (2 points), and the aforementioned fluency task. Lastly, orientation to time and place is evaluated (6 points). (See, for example the World Wide Web atmocatest.org.)

In some embodiments, a decrease in the patient rate of transport as compared to the control rate of transport is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% correlates to a MoCA score of about 30, about 29, about 28, about 27, about 26, about 25, about 24, or about 23.

In some embodiment, a decrease in the rate of transport can also be referred to as an increase in the amount of time necessary for one or more cargo molecules to appear and to be detectable in a patient sample. In some embodiments, the decrease in the rate of transport in a patient as compared to the rate of transport in a control subject by at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days correlates to a MoCA score of about 30, about 29, about 28, about 27, about 26, about 25, about 24, or about 23.

In some embodiments, a patient rate of transport of about 4 days to about 14 days, correlates to a MoCA score of 30 to 23. In some embodiments, a patient rate of transport of about 4 days correlates to a MoCA score of about 30, 29, or 28. In some embodiments, a patient rate of transport of about 7 days or 8 days correlates to a MoCA score of about 27. In some embodiments, a patient rate of transport of about 8 days to about 12 days, or about 8 days, about 9 days, about 10 days, about 11 days, or about 12 days, correlates to a MoCA score of about 26. In some embodiments, a patient rate of transport of about 12 days correlates to a MoCA score of about 25. In some embodiments, a patient rate of transport of about 13 days correlates to a MoCA score of about 24. In some embodiments, a patient rate of transport of about 14 days correlates to a MoCA score of about 23. In some embodiments, the cargo molecule whose rate of transport is decreased and which correlates with the MoCA score is proenkephalin-A.

In some embodiments, a patient rate of transport of about 4 days to about 14 days, correlates to a MoCA score of 30 to 23. In some embodiments, a patient rate of transport of about 4 days or about 5 days correlates to a MoCA score of about 30, 29, or 28. In some embodiments, a patient rate of transport of about 9 days to about 11 days, or about 9 days, about 10 days, or about 11 days, correlates to a MoCA score of about 27. In some embodiments, a patient rate of transport of about 10 days to about 15 days, or about 10 days, about 11 days, about 12 days, about 13 days, about 14 days, or about 15 days correlates to a MoCA score of about 26. In some embodiments, a patient rate of transport of about 16 days correlates to a MoCA score of about 25. In some embodiments, a patient rate of transport of about 17 days or about 18 days correlates to a MoCA score of about 24. In some embodiments, a patient rate of transport of about 18 days or about 19 days correlates to a MoCA score of about 23. In some embodiments, the cargo molecule whose rate of transport is decreased and which correlates with the MoCA score is galanin.

Parkinson's Disease vs. Amyotrophic Lateral Sclerosis: Methods for Distinguishing Between PD and ALS

In some embodiments, the present disclosure provides methods for differentiating between Parkinson's disease (PD) and amyotrophic lateral sclerosis (ALS) in a patient. The method includes a) examining a plurality of patient samples obtained over one or more timepoints from said patient, wherein said samples comprise isotope labeled cargo molecules; b) quantifying the rate of transport of one or more cargo molecules into said samples or in said samples; c) comparing the rate of transport of said one or more cargo molecules into said patient samples or in said patient samples to the rate of transport of said one or more cargo molecules into said control subject samples or in said control subject samples, and d) detecting a difference in the rate of transport of at least two or more cargo molecules into said patient samples or in said patient samples as compared to the rate of transport of one or more cargo molecules into said control subject samples or in said control subject samples, wherein (i) a decrease in the rate of transport of alpha-synuclein into said patient samples or in said patient samples as compared to the rate of transport of alpha-synuclein into said control subject samples or in said control subject samples and (ii) no change in the rate of transport of neuregulin-1 into said patient samples or in said patient samples as compared to the rate of transport of neuregulin-1 into said control subject samples or in said control subject samples is indicative of PD, and wherein (i) a decrease in the rate of transport of neuregulin-1 into said patient samples or in said patient samples as compared to the rate of transport of neuregulin-1 into said control subject samples or in said control subject samples and (ii) no change in the rate of transport of alpha-synuclein into said patient samples or in said patient samples as compared to the rate of transport of alpha-synuclein into said control subject samples or in said control subject samples, is indicative of ALS.

In some embodiments, a plurality of samples includes one or more samples.

In some embodiments, the method further comprises a step of administering an isotope-labeled substrate to said patient for a period of time sufficient for said isotope-labeled substrate or isotope label to be detectable in said patient sample, wherein administration is prior to step a) of the method. Such methods are described in detail herein below. In general, as more fully described below, patients drink isotope-labeled water (e.g., heavy water, D₂O or ²H₂O) over a period of days, generally seven, during which time the substrate is synthesized (i.e., incorporated) into the proteins of the body, including cargo molecules. By sampling at a later time point (generally several weeks later, from 14 to 21 days) and calculating the ratio of isotope-labeled cargo molecules (biomarkers) as a fraction of total cargo molecules, the rate of transport can be calculated and then compared to control subjects. In some embodiments, the timepoint is taken at 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, or more days post isotope-labeled substrate. In some embodiments, the timepoint is taken at 14 days, 15 days, 16 days, 17 days, 18 days, 19 days, 20 days, or 21 days post isotope-labeled substrate.

According to the method of differentiating between PD and ALS, a decrease in the rate of transport of alpha-synuclein in patient samples as compared to the rate of transport of alpha-synuclein in control subject samples in combination with no change in the rate of transport of neuregulin-1 in patient samples as compared to the rate of transport of neuregulin-1 in control subject samples is indicative of the patient having PD.

According to the method of differentiating between PD and ALS, a decrease in the rate of transport of neuregulin-1 in patient samples as compared to the rate of transport of neuregulin-1 in control subject samples in combination with no change in the rate of transport of alpha-synuclein in patient samples as compared to the rate of transport of alpha-synuclein in said control subject samples, is indicative of the patient having ALS.

The rate of transport can be measured by employing the isotope measurement methods known in the art or those described in detail herein below. A decrease in the rate of transport can also be referred to as an increase in the time necessary for one or more cargo molecules to be transported into a patient sample. In some embodiments, the decrease is a decrease in the patient rate of transport as compared to the control rate of transport of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

The rate of transport can be measured by employing the isotope measurement methods known in the art or those described in detail herein below. In some embodiments, a decrease in the rate of transport is referred to as an increase in the amount of time necessary for one or more cargo molecules to appear and to be detectable in a patient sample. In some embodiments, the decrease in the rate of transport is an increase in the amount of time necessary for one or more cargo molecules to appear and to be detectable a patient sample as compared to the amount of time necessary for one or more cargo molecules to appear and to be detectable a control subject sample of at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, or at least about a month.

In some embodiments, a patient rate of transport of alpha-synuclein into a patient sample of about 4 days to about 14 days and a patient rate of transport of neuregulin-1 into a patient sample of about 2 days to about 3 days is indicative of PD in the patient. In some embodiments, a patient rate of transport of alpha-synuclein into a patient sample of about 4 days, about 5 days, about 6 days, about 7 days, about 8 days, about 9 days, about 10 days, about 11 days, about 12 days, about 13 days, or about 14 days and a patient rate of transport of neuregulin-1 into a patient sample of about 2 days or about 3 days is indicative of PD in the patient.

In some embodiments, a patient rate of transport of neuregulin-1 into a patient sample of about 7 days to about 9 days and a patient rate of transport of alpha-synuclein into a patient sample of about 2 days to about 3 days is indicative of ALS in the patient. In some embodiments, a patient rate of transport of neuregulin-1 into a patient sample of about 7 days, or about 8 days, or about 9 days and a patient rate of transport of alpha-synuclein into a patient sample of about 2 days or about 3 days is indicative of ALS in the patient.

### Methods for Monitoring Treatment Regiment in Parkinson's Disease

In some embodiments, the present disclosure provides methods for monitoring a treatment regimen in Parkinson's disease (PD) in a patient being treated for PD with a drug. The methods include a) examining a plurality of patient samples obtained over multiple timepoints from said patient, wherein said samples comprise isotope labeled cargo molecules; b) quantifying the rate of transport of one or more cargo molecules into said patient samples or in said patient samples; c) comparing the rate of transport of said one or more cargo molecules into said patient samples or in said patient samples to the rate of transport of said one or more cargo molecules into said PD patient samples, wherein detecting a reduction in the decrease in the rate of transport of said one or more cargo molecules into said patient samples or in said patient samples as compared to the rate of transport of one more cargo one or more cargo molecules into said PD subject samples or in said PD subject samples, wherein said PD subject samples are (i) from a subject not being treated or (ii) from the same patient prior to beginning treatment is indicative of the therapeutic efficacy of the treatment.

In some embodiments, a plurality of samples includes one or more samples.

In some embodiments, the method further comprises a step of administering an isotope-labeled substrate to said patient for a period of time sufficient for said isotope-labeled substrate or isotope label to be detectable in said patient sample, wherein administration is prior to step a) of the method prior to step a) of the method. Such methods are described in detail herein below. In general, as more fully described below, patients drink isotope-labeled water (e.g., heavy water, D₂O or ²H₂O) over a period of days, generally seven, during which time the substrate is synthesized (i.e., incorporated) into the proteins of the body, including cargo molecules. By sampling at a later time point (generally several weeks later, from 14 to 21 days) and calculating the ratio of isotope-labeled cargo molecules (biomarkers) as a fraction of total cargo molecules, the rate of transport can be calculated and then compared to control subjects. In some embodiments, the timepoint is taken at 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, or more days post isotope-labeled substrate. In some embodiments, the timepoint is taken at 14 days, 15 days, 16 days, 17 days, 18 days, 19 days, 20 days, or 21 days post isotope-labeled substrate.

According to the method of monitoring a treatment regimen in PD, the one or more cargo molecules includes brain-derived neurotrophic factor (BDNF). The rate of transport can be measured by employing the isotope measurement methods known in the art or those described in detail herein below. A decrease in the rate of transport can also be referred to as an increase in the time necessary for one or more cargo molecules to be transported into a patient sample. In some embodiments, the decrease is a decrease in the patient rate of transport as compared to the control rate of transport of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

The rate of transport can be measured by employing the isotope measurement methods known in the art or those described in detail herein below. A decrease in the rate of transport can also be referred to as an increase in the amount of time necessary for one or more cargo molecules to appear and to be detectable in a patient sample. In some embodiments, the decrease in the rate of transport is an increase in the amount of time necessary for one or more cargo molecules to appear and to be detectable in a patient sample as compared to the amount of time necessary for one or more cargo molecules to appear and to be detectable in a control subject sample of at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, or at least about a month.

According to the method of monitoring, part of the method includes detecting a reduction in the decrease in the rate of transport of one or more cargo molecules into patient samples as compared to the rate of transport of one more cargo one or more cargo molecules into PD subject samples, wherein the PD subject samples are (i) from a subject not being treated or (ii) from the same patient prior to beginning treatment is indicative of the therapeutic efficacy of the treatment. In some embodiments, the reduction in the decrease in the patient rate of transport as compared to a subject not being treated or to the same patient prior to beginning treatment is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

A reduction in the decrease is analogous to an increase in the rate of transport. In some embodiments, the increase in the patient rate of transport as compared to a subject not being treated or to the same patient prior to beginning treatment is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

Treatment can include any pharmaceutical compositions as described herein (see, “Pharmaceutical Compositions for Treatment” section below) or used for treatment of PD. In some embodiments, the treatment comprises administration of Azilect (also known as rasagiline). In some embodiments, the treatment comprises administration of cholinesterase inhibitors (e.g. donepezil).

### Methods for Developing Pharmacokinetic Treatment Profiles for Parkinson's Disease

In some embodiments, the present disclosure provides methods of developing pharmacokinetic treatment profiles for Parkinson's disease (PD) in a patient being treated for PD. The method includes a) examining a plurality of patient samples obtained over one or more timepoints from said patient, wherein said samples comprise isotope labeled cargo molecules; b) quantifying the rate of transport of at least two or more cargo molecules into said samples or in said samples; c) comparing the rate of transport of said at least two or more cargo molecules into said patient samples or in said patient samples to the rate of transport of said at least two or more cargo molecules into said PD subject samples or in said PD subject samples, wherein detecting a reduction in the decrease in the rate of transport of said one or more cargo molecules into said patient samples or in said patient samples as compared to the rate of transport of one more cargo one or more cargo molecules into said PD subject samples or in said PD subject samples, wherein said PD subject samples are (i) from a subject not being treated or (ii) from the same patient prior to beginning treatment is indicative of the therapeutic efficacy of the treatment; and d) developing a signature profile for the differential rates of transport of said at least two or more cargo molecules.

In some embodiments, the method further comprises a step of administering an isotope-labeled substrate to said patient for a period of time sufficient for said isotope-labeled substrate or isotope label to be detectable in said patient sample, wherein administration is prior to step a) of the method prior to step a) of the method. Such methods are described in detail herein below. In general, as more fully described below, patients drink isotope-labeled water (e.g., heavy water, D₂O or ²H₂O) over a period of days, generally seven, during which time the substrate is synthesized (i.e., incorporated) into the proteins of the body, including cargo molecules. By sampling at a later time point (generally several weeks later, from 14 to 21 days) and calculating the ratio of isotope-labeled cargo molecules (biomarkers) as a fraction of total cargo molecules, the rate of transport can be calculated and then compared to control subjects. In some embodiments, the timepoint is taken at 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, or more days post isotope-labeled substrate. In some embodiments, the timepoint is taken at 14 days, 15 days, 16 days, 17 days, 18 days, 19 days, 20 days, or 21 days post isotope-labeled substrate.

According to the method of developing pharmacokinetic treatment profiles for Parkinson's disease (PD, the one or more cargo molecules includes brain-derived neurotrophic factor (BDNF).

The rate of transport can be measured by employing the isotope measurement methods known in the art or those described in detail herein below. A decrease in the rate of transport can also be referred to as an increase in the time necessary for one or more cargo molecules to be transported into a patient sample. In some embodiments, the decrease is a decrease in the patient rate of transport as compared to the control rate of transport of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

A decrease in the rate of transport can also be referred to as an increase in the amount of time necessary for one or more cargo molecules to be detectable in a patient sample. In some embodiments, the decrease in the rate of transport is an increase in the amount of time necessary for one or more cargo molecules to be detectable in a patient sample as compared to the amount of time necessary for one or more cargo molecules to be detectable in a control subject sample of at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, or at least about a month.

According to the method of developing pharmacokinetic treatment profiles for Parkinson's disease (PD includes detecting a reduction in the decrease in the rate of transport of one or more cargo molecules into patient samples as compared to the rate of transport of one or more cargo molecules into PD subject samples, wherein PD subject samples are (i) from a subject not being treated or (ii) from the same patient prior to beginning treatment, is indicative of the therapeutic efficacy of the treatment.

In some embodiments, the reduction in the decrease in the patient rate of transport as compared to a subject not being treated or to the same patient prior to beginning treatment is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. A reduction in the decrease is analogous to an increase in the rate of transport. In some embodiments, the increase in the patient rate of transport as compared to a subject not being treated or to the same patient prior to beginning treatment is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

Developing pharmacokinetic treatment profiles for the differential rates of transport of at least two or more cargo molecules can include quantifying rates of transport for each cargo molecule, comparing transport rates for each cargo molecule to clinical diagnostic measurements (including Hoehn & Yarn Scale, UPDRS scale and the MoCA scale, as described herein and below, as well as other clinical diagnostic scales), comparing transport rates for each cargo molecule to the transport rates for a subject not being treated or to the transport rates for the same subject prior to beginning treatment and using any or all of this information to develop a profile regarding the information for one or more cargo molecules. In some embodiments, 1, 2, 3, 4, 5, 6, 7, 8, 9, or 10 or more cargo molecule profiles are developed.

In some embodiments, the development of pharmacokinetic treatment profiles involves determining a decrease in the rate of transport that correlates with a Hoehn & Yahr scale score (Hoehn, M. and Yahr M., “Parkinsonism: onset, progression and mortality” Neurology, 17 (5): 427-442 (1967)). The Hoehn & Yarn scale score is a commonly used system for describing the progress of Parkinson's disease symptoms of Parkinson's disease progress published in 1967 and included stages 1 through 5. Since then, the scale has been modified with the addition of stages 1.5 and 2.5 to help describe the intermediate course of the disease. A summary of the Hoehn & Yarn scale is provided in Table 1 above.

In some embodiments, the development of pharmacokinetic treatment profiles involves determining a decrease in the rate of transport that correlates with a UPDRS Part III score. The unified Parkinson's disease rating scale (UPDRS) is used to follow the longitudinal course of Parkinson's disease. The UPDRS rating scale is another commonly used scale in the clinical study of Parkinson's disease. (Ramaker, C., et al., “Systematic evaluation of rating scales for impairment and disability in Parkinson's disease”, Movement Disorders, 17 (5): 867-876 (2002)). The UPDRS scale is titled; (1) nonmotor experiences of daily living (13 items), (2) motor experiences of daily living (13 items), (3) motor examination (18 items), and (4) motor complications (six items) and each subscale now has 0-4 ratings, where 0=normal, 1=slight, 2=mild, 3=moderate, and 4=severe. A summary of the UPDRS scale is provided in Table 2 above.

In some embodiments, the development of pharmacokinetic treatment profiles involves determining a decrease in the rate of transport that correlates with a MoCA score. The Montreal Cognitive Assessment (MoCA) was created in 1996 has been validated and used to evaluate cognitive abilities in clinical settings (Nasreddine Z. S., et al., “The Montreal Cognitive Assessment (MoCA): A Brief Screening Tool For Mild Cognitive Impairment”, Journal of the American Geriatrics Society 53:695-699 (2005)).

The MoCA assesses multiple cognitive domains. The short-term memory recall task (5 points) involves two learning trials of five nouns and delayed recall after approximately 5 minutes. Visuospatial abilities are assessed using a clock-drawing task (3 points) and a three-dimensional cube copy (1 point). Multiple aspects of executive functions are assessed using an alternation task adapted from the trail-making B task (1 point), a phonemic fluency task (1 point), and a two-item verbal abstraction task (2 points). Attention, concentration and working memory are evaluated using a sustained attention task such as target detection using tapping (1 point), a serial subtraction task (3 points), and digits forward and backward (1 point each). Language is assessed using a three-item confrontation naming task with low-familiarity animals (lion, camel, rhinoceros; 3 points), repetition of two syntactically complex sentences (2 points), and the aforementioned fluency task. Lastly, orientation to time and place is evaluated (6 points). (See, for example the World Wide Web atmocatest.org.)

Treatment can include any pharmaceutical formulations as described herein see, “Pharmaceutical Compositions for Treatment” section below) or used for treatment of PD. In some embodiments, the treatment includes drugs currently in clinical trials for ALS and/or PD. In some embodiments, the treatment comprises administration of rasagiline (Azilect). In some embodiments, the treatment comprises administration of cholinesterase inhibitors (e.g., donepezil). In one embodiment, pharmacokinetic treatment profiles for Parkinson's disease (PD) are determined for rasagiline (Azilect). In one embodiment, pharmacokinetic treatment profiles for Parkinson's disease (PD) are determined for cholinesterase inhibitors (e.g., donepezil). In one embodiment, pharmacokinetic treatment profiles for Parkinson's disease (PD) are determined for microtubule target modulating agents. By “microtubule target modulating agent” or “MTMA” herein is meant an agent that has been previously recognized or proposed to affect the rate of microtubule polymerization and/or depolymerization, and in particular to reduce or slow microtubule instability (i.e., dynamicity).

In one embodiment, the MTMAs are opioids and opioid derivatives. There are four broad classes of opioids: endogenous opioid peptides, produced in the body; opium alkaloids, such as morphine (the prototypical opioid) and codeine; semi-synthetic opioids such as heroin and oxycodone; and fully synthetic opioids such as pethidine and methadone that have structures unrelated to the opium alkaloids.

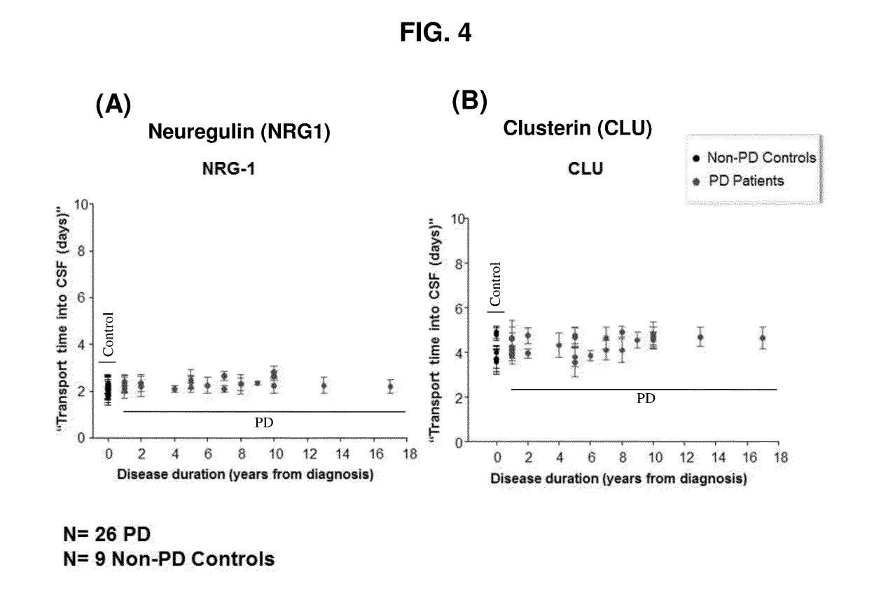

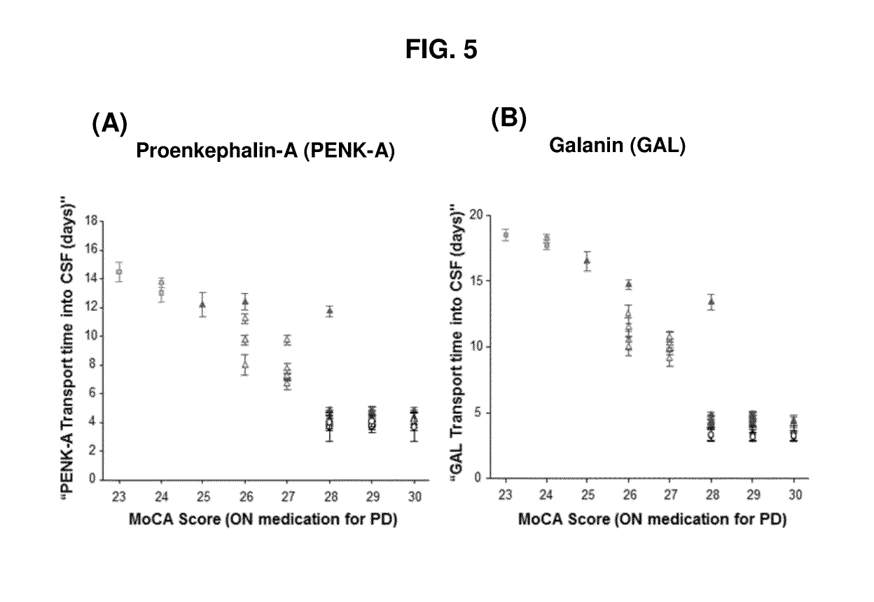

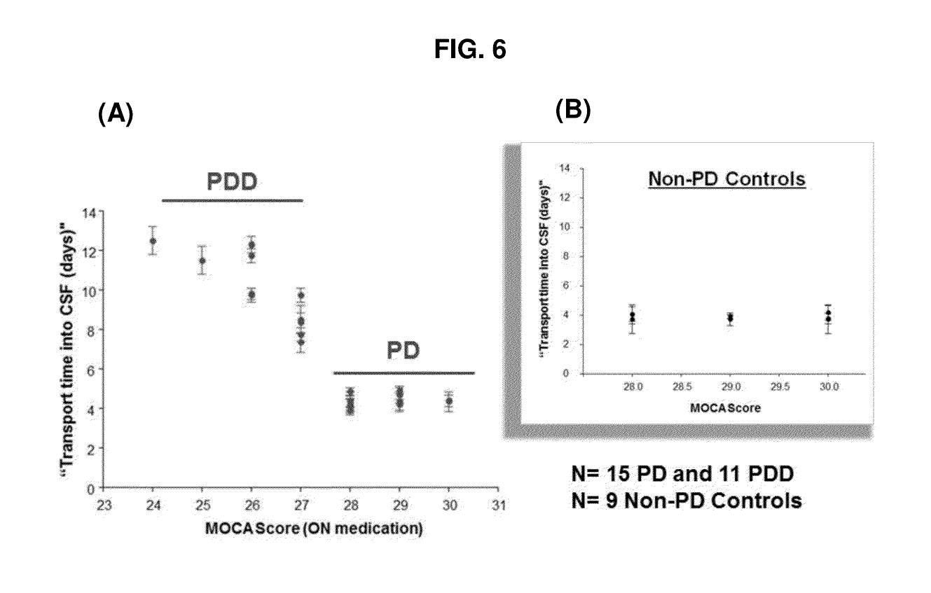

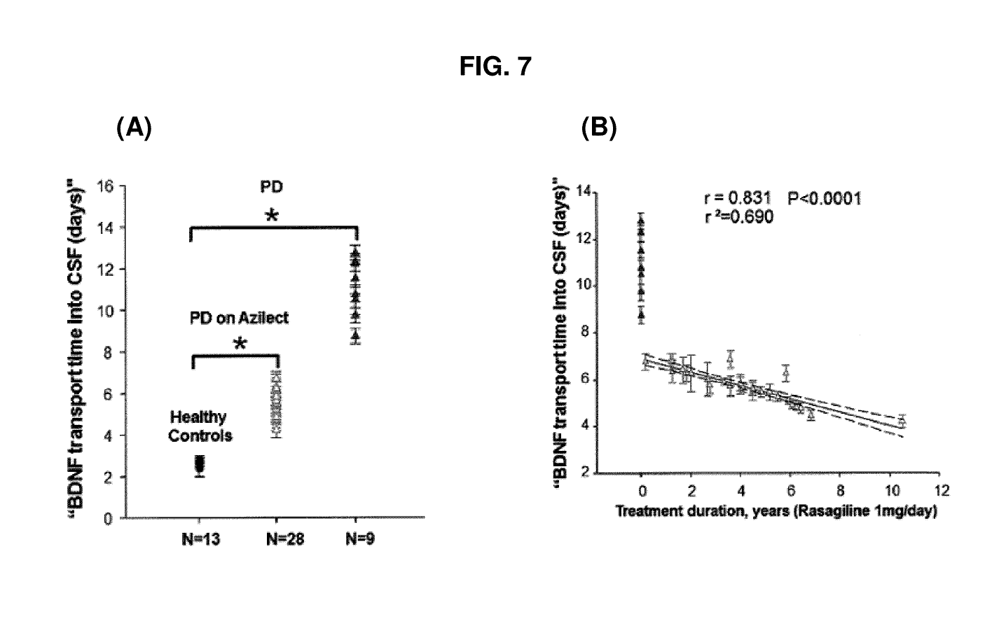

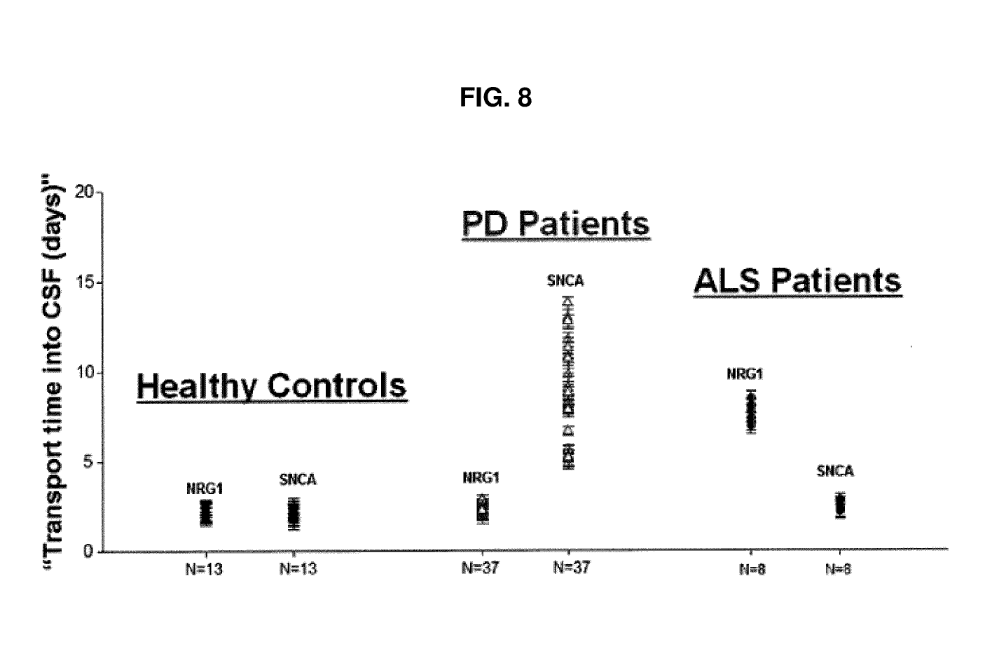

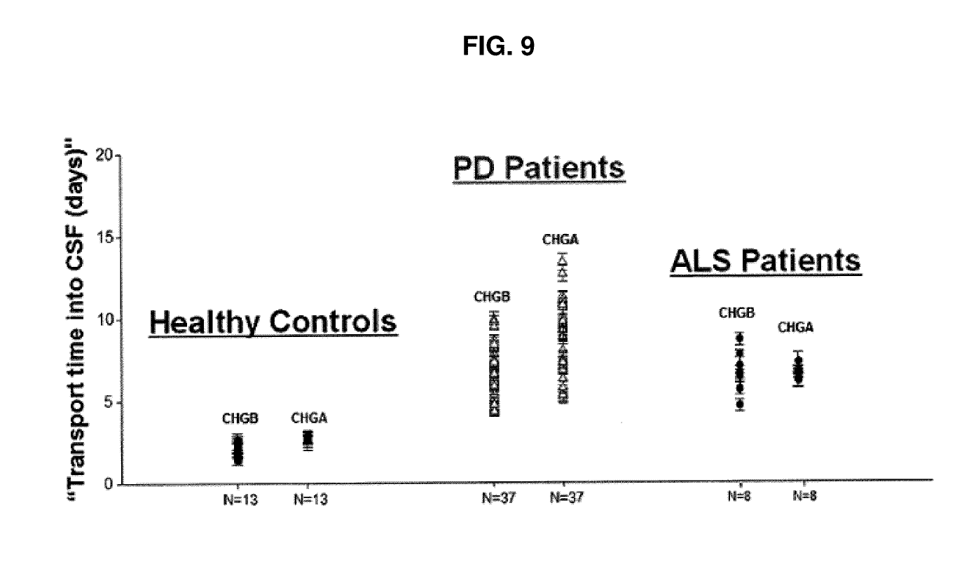

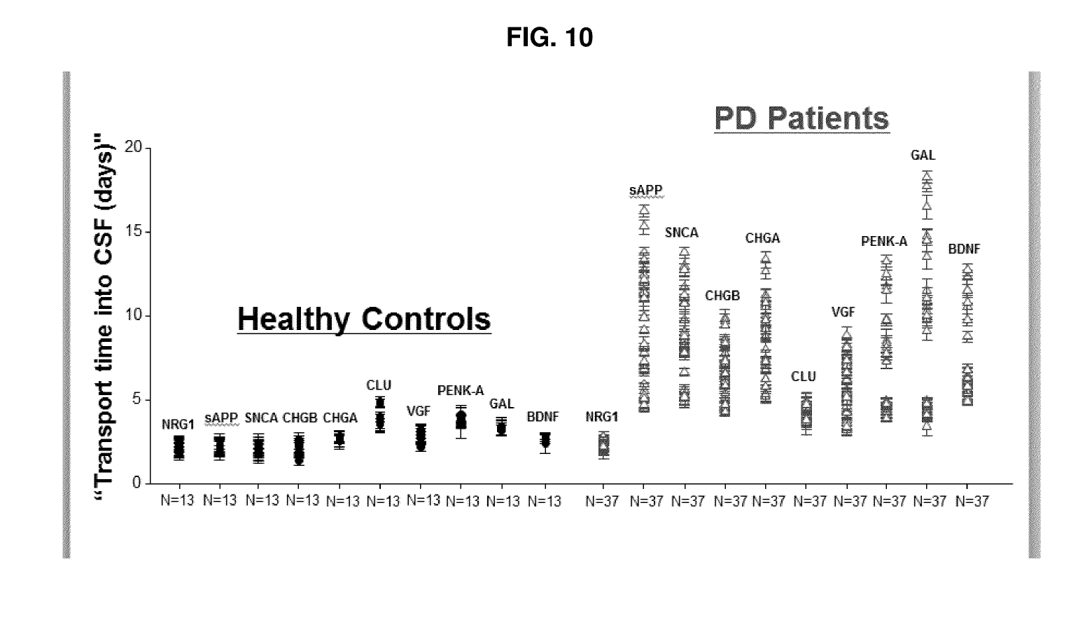

In one embodiment, the MTMAs are opium alkaloids. In one embodiment, the opium alkaloid is noscapine or noscapine derivatives such as outlined in U.S. Pat. No. 6,376,516, hereby incorporated by reference in its entirety. Noscapine is an opium alkaloid that lacks analgesic or anticonvulsant activity, and contrary to other opioids (e.g., morphine) is not a narcotic or an addicting compound. Furthermore, in contrast to other microtubule-interacting agents such as paclitaxel, nocodazole, vinblastine and colchicine, noscapine modifies microtubule dynamics without affecting total tubulin polymer mass and without altering the steady-state dimer/polymer equilibrium of microtubule assembly both in vitro and in living cells. Noscapine penetrates the blood brain barrier and has long half-life in CNS tissue (brain and spinal cord) and PNS too. Therefore, we identified noscapine as a potential microtubule-interactive chemotherapeutic agent for CNS and PNS disorders associated with cytoskeletal abnormalities such as altered microtubule dynamics as we have discovered (see FIGS. 4-7). Noscapine is currently available for human use as a cough suppressant. Other opium alkaloids useful as MTMAs are the phenanthrenes, isoquinolines and papaverine.

In addition, the cannabinoids find use in screening alone or in combination with other agents. Cannabinoids are a group of chemicals which activate the body's endogenous cannabinoid receptors, including CB1 and CB2 receptor. Currently, there are three general types of cannabinoids: herbal cannabinoids occur uniquely in the cannabis plant; endogenous cannabinoids are produced in the bodies of humans and other animals; and synthetic cannabinoids are similar compounds produced in the laboratory. Suitable agents include, but are not limited to: anandamide and analogs of anandamide, docosatetraenylethanolamide and homo-γ-linoenylethanolamide; endocannabinoids such as 2-arachidonoylglycerol (2-AG), palmitoyl ethanolamide and oleamide; tetrahydrocannabinol (THC), particularly Marinol (Δ⁹-THC), cannabidiol (CDB); cannabinol (CBN); Cannabigerol; Cannabichromene; Cannabicyclol; Cannabivarol; Tetrahydrocannabivarin; Cannabidivarin; Cannabichromevarin; Cannabigerovarin; Cannabigerol Monoethyl Ether, CP-55940; HU-210 100; SR-144526; and Nabilone.

Other agents are those that target the intracellular concentration of ions, particularly calcium and sodium. Intracellular concentration of calcium is known to be involved in microtubule formation and stability, and thus agents that modulate intracellular calcium (particularly by decreasing intracellular calcium concentrations) are of particular interest for screening.

There are three main types of ligand-gated ion channels (ionotropic receptors) that are involved in the L-glutamate pathway, a major excitatory neurotransmitter. These are the NMDA, AMPA and kainate receptors, each of which pharmacokinetic treatment profiles for Parkinson's disease (PD) can be developed according to the present disclosure.

NMDA Receptor Antagonists

The NMDA receptor was first identified by the selective activation by N-methyl-D-aspartate (NMDA). NMDA receptors are composed of assemblies of NR1 subunits and NR2 subunits, which can be one of four separate gene products (NR2A-D). Expression of both subunits is required to form functional channels. The glutamate binding domain is formed at the junction of NR1 and NR2 subunits (hence the need for both subunits to be expressed). In addition to glutamate, the NMDA receptor requires a co-agonist, glycine, to bind to allow the receptor to function. The glycine binding site is found on the NR1 subunit. The NR2B subunit also possesses a binding site for polyamines, regulatory molecules that modulate the functioning of the NMDA receptor. In addition to the glutamate (NMDA) binding site, there are also multiple binding sites on the NMDA receptor for modulatory compounds. Efficient NMDA receptor activation requires not only NMDA but also glycine. Activation can also be modulated by the binding of polyamines Each of the binding sites (glutamate, glycine, polyamine) has been used as a potential target for the development of both receptor and sub-type selective compounds.

NMDA inhibitors can be either competitive or non-competitive inhibitors, and can bind to any of the binding sites. Thus, suitable NMDA receptor antagonists include, but are not limited to, Amantadine; ketamine; dextromethorphan (3-methoxy-17-methyl-9(alpha), 13(alpha), 14(alpha)-morphinana hydrobromide monohydrate); Dizocilipine (also known as MK-801); AP-7 (2-amino-7-phosphonoheptanoic acid); APV (also called AP-5; 2-amino-5-phosphonovalerate; DCKA (5,7-dichlorokyneurenic acid; acts at the glycine site), harkoseride (acetamido-N-benzyl-3-methoxypropionate and its metabolite, H-209); homoquinolinic acid, (R)-AP5; (R)-CPP-ene; PBPD; memantine; ketamine; L-701-324; L-689, 560; GV196771A; Ro 25-6981; ifenprodil; Co-101676; GW468816 (glycine site antagonist).

Of particular interest is dizolcipine; dizolcipine has been identified as a calcium channel blocker which decreases the excessive influx of calcium into neurons through the ionic channel NMDA-receptor. It is also classified as a competitive antagonist of the glutamatergic NMDA-receptor subtype and penetrates the blood brain barrier. Glutamate-induced excitotoxicity is complex and multifactorial, but is a major component of the terminal events mediating neuronal injury and death. It involves excessive influx of calcium through the NMDA-receptor.

Modulators of Oxidative Stress

In addition to MTMAs and receptor antagonists, other agents include those that effect oxidative stress in motoneurons. These include general antioxidants such as Vitamin E, procysteine, N-acetylcysteine, lipoic acid, and various types of nitrones.

Microglia Activation

There are several candidate anti-inflammatory drugs, including, but not limited to, minocycline and thalidomide for which pharmacokinetic treatment profiles for Parkinson's disease (PD) could be developed according to the methods of the present disclosure.

Miscellaneous Agents

In addition, anti-glutamate agents, other anti-inflammatory agents and other anti-convulsants can all be tested. Of course, the invention is not limited by any particular compound in any particular class of compounds. Any compound or any combination of compounds is envisioned for use in the methods of the present invention, in particular any compound contemplated for us in a Parkinson's disease treatment regimen.

### Evaluation of Drug Treatments, Including Drug Candidates

One particularly useful embodiment of the present invention is the use of the invention to test and/or monitor the effects of drugs, including drug candidates. “Drugs” in this context include drugs already approved for use in humans, both for motoneuronal diseases as well as any other approved drugs, including for example **R drug** approved for use in PD patients (e.g., drugs undergoing clinical trials). Drug candidates in this context are drugs that are drugs in development, either preclinical or clinical testing, that may find use in the treatment of motoneuronal diseases. The ability to have physical, subjective biomarker evaluation of drugs that can alter the rate of transport of cargo molecules, shown to be symptomatic and/or causative in these diseases is a valuable tool for drug screening and/or testing. Thus, for example, using the present invention in animal models of PD and/or ALS for drug screening, based on the cargo biomarker molecules allows for rapid and subjective analysis. This is particularly useful in these diseases where the evaluation of cognitive and/or physical manifestations of disease are more difficult. Similarly, for use in PD and/or ALS patients, the ability to similarly evaluate a subjective biomarker panel (including one or more of the biomarker cargo molecules discussed herein) for patients who are not yet definitively diagnosed, have been diagnosed and who are contemplating drug treatments, those patients already on drugs, etc. is very useful. This is particularly useful in these diseases where symptom onset can be very slow, or difficult to evaluate in short periods of time.

In some embodiments, the rate of transport for patients can be initially evaluated in the absence of the drug, the drug is administered for some period of time (from as little as 1 week to 1, 2, 3 to 6 months) and then the patient is retested. A difference in the rate of transport of one or more cargo molecules, particularly increases in the rate of transport or changes in the rate of transport more closely resemble a control rate of transport, can show drug efficacy. In some embodiments, the patient may already be on one drug and a second is evaluated. In some embodiments, the patient may already be on one drug and then switched to a different drug in order to optimize treatment or evaluate in order to identify a more effective treatment.

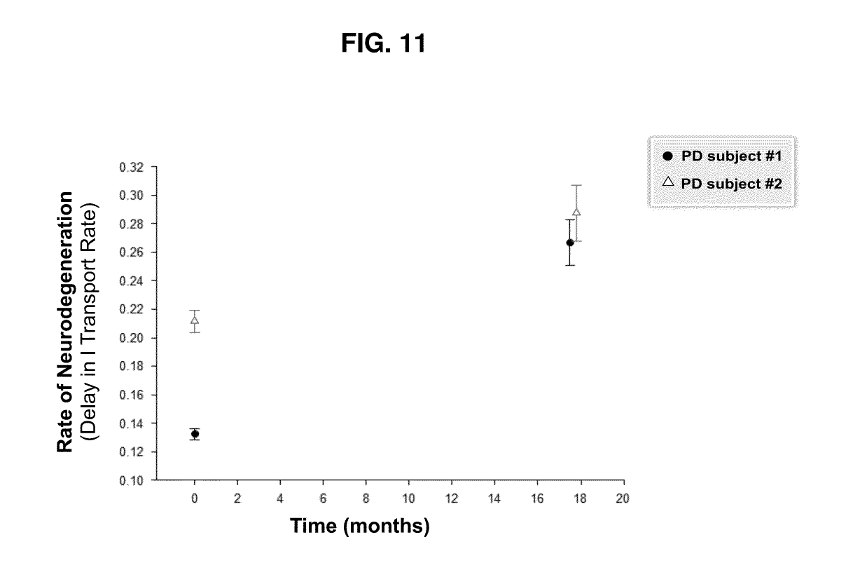

In some embodiments, the patient may be on a drug initially and the testing is done to see whether the drug is slowing progression; e.g. the rate of change in the rate of transport is measured. That is, a change in the rate of decreasing transport can be indicative of drug efficacy or lack thereof. Accordingly, the present methods can be used to evaluate prognosis and disease progression, see FIG. 11.

In one embodiment, the drug is a drug in clinical trials. In one embodiment, the drug is a drug in clinical trials for ALS. In one embodiment, the drug is a drug in clinical trials for PD. In one embodiment, the drug is rasagiline (Azilect). In one embodiment, is one or more cholinesterase inhibitors (e.g., donepezil).

### Administering Isotope-Labeled Precursor(s)

In some embodiments, the method further comprises a step of administering an isotope-labeled substrate to said patient for a period of time sufficient for said isotope-labeled substrate or isotope label to be detectable in said patient sample, wherein administration is prior to step a) of the method prior to step a) of the method. Such methods are described in detail herein below. Methods for administering isotope labeled precursor(s) are described in detail below, as well as in U.S. Publication No. 2014/0274785 and PCT Publication No. WO 2009/143365, both of which are incorporated by reference herein in their entireties.

In the embodiments of the method which involve the administration of isotope-labeled precursors to living systems (i.e., patients or subjects) or to obtain samples from living systems to which isotope labeled precursors have been obtained. The phrases “living system” or “living systems” include but are not limited to, cells, cell lines, animal models of disease, guinea pigs, rabbits, dogs, cats, other pet animals, mice, rats, non-human primates, and humans. In particular, living systems refer to human patients or subjects.

In some embodiments, the first step of the method involves administering an isotope-labeled precursor molecule to a living system. Modes of administering isotope-labeled precursor molecules may vary, depending upon the absorptive properties of the isotope-labeled precursor molecule and the specific biosynthetic pool into which each compound is targeted. Precursors may be administered to organisms, including experimental animals and humans directly for in vivo analysis.

Generally, an appropriate mode of administration is one that produces a steady state level of precursor within the biosynthetic pool and/or in a reservoir supplying such a pool for at least a transient period of time. Intravascular or oral routes of administration are commonly used to administer such precursors to organisms, including humans. Other routes of administration, such as subcutaneous or intra-muscular administration, optionally when used in conjunction with slow release precursor compositions, are also appropriate. Compositions for injection are generally prepared in sterile pharmaceutical excipients. The selection of which route to administer an isotope-labeled precursor molecules is within the skill of the art.

The isotope-labeled precursor molecule may be a stable isotope or radioisotope. Isotope labels that can be used include, but are not limited to, ²H, ¹³C, ¹⁵N, ¹⁸O, ³H, ¹⁴C, ³⁵S, ³²P, ¹²⁵I, ¹³¹I, or other isotopes of elements present in organic systems.

In one embodiment, the isotope label is ²H.

The precursor molecule may be any molecule having an isotope label that is incorporated into the “monomer” or “subunit” of interest, or it can be the monomer itself. Isotope labels may be used to modify all precursor molecules disclosed herein to form isotope-labeled precursor molecules. “Isotope labeled substrate” includes any isotope-labeled precursor molecule that is able to be incorporated into a molecule of interest in a living system. Examples of isotope labeled substrates include, but are not limited to, ²H₂O, ³H₂O, ²H-glucose, ²H-labeled amino acids, ²H-labeled organic molecules, ¹³C-labeled organic molecules, ¹⁴C-labeled organic molecules, ¹³CO₂, ¹⁴CO₂, ¹⁵N-labeled organic molecules and ¹⁵NH₃.

The entire precursor molecule may be incorporated into one or more tubulin dimer subunits. Alternatively, a portion of the precursor molecule may be incorporated into the tubulin dimer subunits.

A protein precursor molecule may be any protein precursor molecule known in the art. These precursor molecules include, but are not limited to, CO₂, NH₃, glucose, lactate, H₂O, acetate, and fatty acids.

Precursor molecules of proteins may also include one or more amino acids. The precursor may be any amino acid. The precursor molecule may be a singly or multiply deuterated amino acid. For example, the precursor molecule may be one or more ¹³C-lysine, ¹⁵N-histidine, ¹³C-serine, ¹³C-glycine, ²H-leucine, ¹⁵N-glycine, ¹³C-leucine, ²H₅-histidine, and any deuterated amino acid. Labeled amino acids may be administered, for example, undiluted or diluted with non-labeled amino acids. All isotope-labeled precursors may be purchased commercially, for example, from Cambridge Isotope Labs (Andover, Mass.).

Protein precursor molecules may also include any precursor for post-translationally or pre-translationally modified amino acids. These precursors include but are not limited to precursors of methylation such as glycine, serine or H₂O; precursors of hydroxylation, such as H₂O or O₂; precursors of phosphorylation, such as phosphate, H₂O or O₂; precursors of prenylation, such as fatty acids, acetate, H₂O, ethanol, ketone bodies, glucose, or fructose; precursors of carboxylation, such as CO₂, O₂, H₂O, or glucose; precursors of acetylation, such as acetate, ethanol, glucose, fructose, lactate, alanine, H₂O, CO₂, or O₂; precursors of glycosylation and other post-translational modifications known in the art.

The degree of labeling present in free amino acids may be determined experimentally, or may be assumed based on the number of labeling sites in an amino acid. For example, when using hydrogen isotopes as a label, the labeling present in C—H bonds of free amino acid or, more specifically, in tRNA-amino acids, during exposure to ²H₂O in body water may be identified. The total number of C—H bonds in each non-essential amino acid is known—e.g., 4 in alanine, 2 in glycine, etc.

The precursor molecule for proteins may be water (e.g., heavy water). The hydrogen atoms on C—H bonds are the hydrogen atoms on amino acids that are useful for measuring protein synthesis from ²H₂O since the O—H and N—H bonds of proteins are labile in aqueous solution. As such, the exchange of ²H-label from ²H₂O into O—H or N—H bonds occurs without the synthesis of proteins from free amino acids. C—H bonds undergo incorporation from H₂O into free amino acids during specific enzyme-catalyzed intermediary metabolic reactions. The presence of ²H-label in C—H bonds of protein-bound amino acids after ²H₂O administration therefore means that the protein was assembled from amino acids that were in the free form during the period of ²H₂O exposure—e.g., that the protein is newly synthesized. Analytically, the amino acid derivative used must contain all the C—H bonds but must remove all potentially contaminating N—H and O—H bonds.

Hydrogen atoms (e.g., deuterium or tritium) from body water may be incorporated into free amino acids. ²H or ³H from labeled water can enter into free amino acids in the cell through the reactions of intermediary metabolism, but ²H or ³H cannot enter into amino acids that are present in peptide bonds or that are bound to transfer RNA. Free essential amino acids may incorporate a single hydrogen atom from body water into the α-carbon C—H bond, through rapidly reversible transamination reactions. Free non-essential amino acids contain a larger number of metabolically exchangeable C—H bonds, of course, and are therefore expected to exhibit higher isotopic enrichment values per molecule from ²H₂O in newly synthesized proteins.

One of skill in the art will recognize that labeled hydrogen atoms from body water may be incorporated into other amino acids via other biochemical pathways. For example, it is known in the art that hydrogen atoms from water may be incorporated into glutamate via synthesis of the precursor α-ketoglutarate in the citric acid cycle. Glutamate, in turn, is known to be the biochemical precursor for glutamine, proline, and arginine. By way of another example, hydrogen atoms from body water may be incorporated into post-translationally modified amino acids, such as the methyl group in 3-methyl-histidine, the hydroxyl group in hydroxyproline or hydroxylysine, and others. Other amino acid synthesis pathways are known to those of skill in the art.

Oxygen atoms (H₂¹⁸O) may also be incorporated into amino acids from ¹⁸O₂ through enzyme-catalyzed reactions (including hydroxyproline, hydroxylysine or other post-translationally modified amino acids). For example, oxygen exchange into the carboxylic acid moiety of amino acids may occur during enzyme-catalyzed reactions. Incorporation of labeled oxygen into amino acids is known to one of skill in the art.

Hydrogen and oxygen labels from labeled water also may be incorporated into amino acids through post-translational modifications. In one embodiment, the post-translational modification already may include labeled hydrogen or oxygen through biosynthetic pathways prior to post-translational modification. In another embodiment, the post-translational modification may incorporate labeled hydrogen, oxygen, carbon, or nitrogen from metabolic derivatives involved in the free exchange-labeled hydrogens from body water, either before or after post-translational modification step (e.g., methylation, hydroxylation, phosphorylation, prenylation, sulfation, carboxylation, acetylation, glycosylation, or other known post-translational modifications).

Protein precursors that are suitable for administration into a subject include, but are not limited to, H₂O, CO₂, NH₃ and HCO₃, in addition to the standard amino acids found in proteins as described, supra.

Water is a precursor of proteins as well as other biological molecules (see U.S. patent application Ser. No. 10/279,399, hereby incorporated by reference in its entirety). As such, labeled water may serve as a precursor in the methods taught herein. “Isotope-labeled water” includes water labeled with one or more specific heavy isotopes of either hydrogen or oxygen. Specific examples of isotope-labeled water include ²H₂O, ³H₂O, and H₂¹⁸O.

H₂O availability is probably never limiting for biosynthetic reactions in a cell (because H₂O represents close to 70% of the content of cells, or >35 molar concentration), but hydrogen and oxygen atoms from H₂O contribute stoichiometrically to many reactions involved in biosynthetic pathways: e.g.: R—CO—CH₂—COOH+NADPH+H₂O→R—CH₂CH₂COOH (fatty acid synthesis).

As a consequence, isotope labels provided in the form of H- or O-isotope-labeled water is incorporated into biological molecules as part of synthetic pathways. Hydrogen incorporation can occur in two ways: into labile positions in a molecule (i.e., rapidly exchangeable, not requiring enzyme catalyzed reactions) or into stable positions (i.e., not rapidly exchangeable, requiring enzyme catalysis). Oxygen incorporation occurs in stable positions.

Some of the hydrogen-incorporating steps from cellular water into C—H bonds in biological molecules only occur during well-defined enzyme-catalyzed steps in the biosynthetic reaction sequence, and are not labile (exchangeable with solvent water in the tissue) once present in the mature end-product molecules. For example, the C—H bonds on glucose are not exchangeable in solution. In contrast, each of the following C—H positions exchanges with body water during reversal of specific enzymatic reactions: C-1 and C-6, in the oxaloacetate/succinate sequence in the Krebs' cycle and in the lactate/pyruvate reaction; C-2, in the glucose-6-phosphate/fructose-6-phosphate reaction; C-3 and C-4, in the glyceraldehyde-3-phosphate/dihydroxyacetone-phosphate reaction; C-5, in the 3-phosphoglycerate/glyceraldehyde-3-phosphate and glucose-6-phosphate/fructose-6-phosphate reactions.

Labeled hydrogen or oxygen atoms from water that are covalently incorporated into specific non-labile positions of a molecule thereby reveals the molecule's “biosynthetic history”—i.e., label incorporation signifies that the molecule was synthesized during the period that isotope-labeled water was present in cellular water.

The labile hydrogens (non-covalently associated or present in exchangeable covalent bonds) in these biological molecules do not reveal the molecule's biosynthetic history. Labile hydrogen atoms can be easily removed by incubation with unlabeled water (H₂O) (i.e., by reversal of the same non-enzymatic exchange reactions through which ²H or ³H was incorporated in the first place), however:

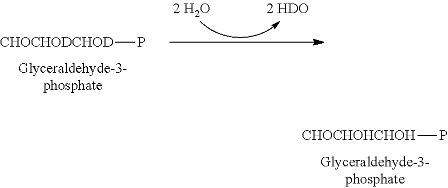

As a consequence, potentially contaminating hydrogen label that does not reflect biosynthetic history, but is incorporated via non-synthetic exchange reactions, can

Analytic methods are available for measuring quantitatively the incorporation of labeled hydrogen atoms into biological molecules (e.g., liquid scintillation counting for ³H; mass spectrometry, laser spectroscopy, NMR spectroscopy or other methods known in the art for ²H and ¹⁸O). For further discussions on the theory of isotope-labeled water incorporation, see, for example, Jungas R L. Biochemistry. 1968 7:3708-17, incorporated herein by reference.

Labeled water may be readily obtained commercially. For example, ²H₂O may be purchased from Cambridge Isotope Labs (Andover, Mass.), and ³H₂O may be purchased, e.g., from New England Nuclear, Inc. “Dueterated water” refers to water incorporating one or more ²H isotopes. In general, ²H₂O is non-radioactive and thus, presents fewer toxicity concerns than radioactive ³H₂O. ²H₂O may be administered, for example, as a percent of total body water, e.g., 1% of total body water consumed (e.g., for 3 liters water consumed per day, 30 microliters ²H₂O is consumed). If ³H₂O is utilized, then a non-toxic amount, which is readily determined by those of skill in the art, is administered.

Relatively high body water enrichments of ²H₂O (e.g., 1-10% of the total body water is labeled) may be achieved relatively inexpensively using the techniques of the invention. This water enrichment is relatively constant and stable as these levels are maintained for weeks or months in humans and in experimental animals without any evidence of toxicity. This finding in a large number of human subjects (>100 persons) is contrary to previous concerns about vestibular toxicities at high doses of ²H₂O. One of the Applicants has discovered that as long as rapid changes in body water enrichment are prevented (e.g., by initial administration in small, divided doses), high body water enrichments of ²H₂O can be maintained with no toxicities. For example, the low expense of commercially available ²H₂O allows long-term maintenance of enrichments in the 1-5% range at relatively low expense (e.g., calculations reveal a lower cost for 2 months labeling at 2% ²H₂O enrichment, and thus 7-8% enrichment in the alanine precursor pool, than for 12 hours labeling of ²H-leucine at 10% free leucine enrichment, and thus 7-8% enrichment in leucine precursor pool for that period).

Relatively high and relatively constant body water enrichments for administration of H₂¹⁸O may also be accomplished, since the ¹⁸O isotope is not toxic, and does not present a significant health risk as a result.

Isotope-labeled water may be administered via continuous isotope-labeled water administration, discontinuous isotope-labeled water administration, or after single or multiple administration of isotope-labeled water administration. In continuous isotope-labeled water administration, isotope-labeled water is administered to an individual for a period of time sufficient to maintain relatively constant water enrichments over time in the individual. For continuous methods, labeled water is optimally administered for a period of sufficient duration to achieve a steady state concentration (e.g., 3-8 weeks in humans, 1-2 weeks in rodents).

In discontinuous isotope-labeled water administration, an amount of isotope-labeled water is measured and then administered, one or more times, and then the exposure to isotope-labeled water is discontinued and wash-out of isotope-labeled water from body water pool is allowed to occur. The time course of delabeling may then be monitored. Water is optimally administered for a period of sufficient duration to achieve detectable levels in biological molecules.

Isotope-labeled water may be administered to an individual or tissue in various ways known in the art. For example, isotope-labeled water may be administered orally, parenterally, subcutaneously, intravascularly (e.g., intravenously, intra-arterially), or intraperitoneally. Several commercial sources of ²H₂O and H₂¹⁸O are available, including Isotec, Inc. (Miamisburg Ohio, and Cambridge Isotopes, Inc. (Andover, Mass.)). The isotopic content of isotope labeled water that is administered can range from about 0.001% to about 20% and depends upon the analytic sensitivity of the instrument used to measure the isotopic content of the biological molecules. In one embodiment, 4% ²H₂O in drinking water is orally administered. In another embodiment, a human is administered 50 mL of ²H₂O orally.

The individual being administered labeled water or which has already been administered labeled water may be a mammal. In one variation, the individual may be an experimental animal including, without limitation, a rodent, primate, hamster, guinea pig, dog, or pig. In variations involving the administering of drugs, drug candidates, drug leads, or combinations thereof, the individual may be a mammal, such as an experimental animal, including an accepted animal model of disease, or a human. In variations involving the administering of food additives, industrial or occupational chemicals, environmental pollutants, or cosmetics, the individual may be any experimental animal such as, without limitation, a rodent, primate, hamster, guinea pig, dog, or pig.

²H₂O Labeling

In general, as more fully described below, patients drink isotope-labeled water (e.g., heavy water, D₂O or ²H₂O) over a period of days, generally seven, during which time the substrate is synthesized (i.e., incorporated) into the proteins of the body, including cargo molecules. By sampling at a later time point (generally several weeks later, from 14 to 21 days) and calculating the ratio of isotope-labeled cargo molecules (biomarkers) as a fraction of total cargo molecules, the rate of transport can be calculated and then compared to control subjects. In some embodiments, the timepoint is taken at 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, or more days post isotope-labeled substrate e. In some embodiments, the timepoint is taken at 14 days, 15 days, 16 days, 17 days, 18 days, 19 days, 20 days, or 21 days post isotope-labeled substrate. In some embodiments, the timepoint is determined based on the cargo protein half-life in order to maximize cargo protein detection.

In practicing the method of the invention, in some embodiments, patients can be administered heavy water (²H₂O) according to any method known in the art such that the heavy water is incorporated into cargo molecules which can then be obtained from any samples as described herein. Any of the cargo molecules described herein can incorporate heavy water. In some embodiments, the cargo molecules which incorporate ²H from heavy water include alpha-synuclein, chromogranin B, chromogranin A, sAPP, VGF, neuregulin-1, clusterin. proenkephalin-A, galanin, and/or brain-derived neurotrophic factor (BDNF) and any combinations thereof according the methods described herein.

Labeling.

The study group consisted of symptomatic PD patients, all on standard anti-PD medications, and 6 non-PD volunteers. PD diagnosis was based on clinical evaluation and UK Parkinson's Disease Society Brain Bank Clinical Diagnostic Criteria (Hughes A J, Daniel S E, Kilford L, Lees A J., J Neurol Neurosurg Psychiatry, 55(3):181-184 (1992)). Busch R, et al. Measurement of protein turnover). Unified PD Rating Scale, PART III, and Hoehn and Yahr scores were obtained for each subject. None of the patients had any atypical features or evidence of more widespread neurological disease. The 6 non-PD controls included 3 healthy volunteers and 3 individuals with well-treated HIV infection; the latter were on combination antiretroviral therapy with long-term plasma viral suppression, had normal CSF white blood cell counts, and CSF/plasma albumin ratios indicated absence of local inflammation or abnormal blood-brain barrier permeability. Eligible subjects were instructed to drink 50 ml of 70% ²H₂O (Isotec-Sigma) 3 times per day, leaving at least 3 hours between doses, for a period of 7 consecutive days. Compliance to the experimental protocol was monitored by the clinical staff through collection of blood plasma samples for body water enrichment. 4 LPs were conducted in 6 controls and 1 PD patient (days 2-15 and 21-43), and a single LP was conducted in the other 11 PD subjects (days 15, 21, 22, or 23), after starting ²H₂O administration (7 days labeling protocol).

Processing of Cargo Proteins for GC/MS Analysis.

Purified cargo proteins were hydrolyzed by treatment with 6N HCl for 16 hours at 110° C. Protein derived amino acids were derivatized to pentafluorobenzyl derivatives, and ²H incorporation in alanine released from total vesicular proteins was measured by GC/MS, as described elsewhere (Busch R, et al., Biochim Biophys Acta., 1760(5):730-44 (2006)). ²H enrichment was calculated as the percent increase over the natural abundance of alanine derivative present as the (M+1) mass isotopomer (EM1) (Busch R, et al., Biochim Biophys Acta., 1760(5):730-44 (2006)). Calculations. The fraction of newly synthesized alanine in each sample was calculated as the ratio of the measured EM1 value to the maximal value expected at the measured body water enrichment, which was calculated by mass isotopomer distribution analysis, as described in detail elsewhere (Busch R, et al., Biochim Biophys Acta., 1760(5):730-44 (2006)). This value was taken to represent fractional protein synthesis.

Statistics.

The statistical significance of CSF secretion rates of selected cargo molecules was assessed by 1-way ANOVA with Tukey post-hoc testing. A P value less than 0.05 was considered statistically significant. Software for statistics included SigmaStat3.0 and Microsoft Excel 2003.

See, Fanara et. al., JCI Clin Invest. doi:10.1172/JCI64575; incorporated herein by reference in its entirety.

### Sample Timepoints

The frequency of biological sampling can vary depending on different factors. Such factors include, but are not limited to, ease and safety of sampling, synthesis and breakdown/removal rates of the proteins, and the half-life of a therapeutic candidate agent administered to a cell, animal, or human. Samples can be taken over a time course of minutes, hours, days, weeks or months. Samples for use in any of the methods described herein can be taken about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, or about a month or more.

A variety of biological samples can be taken in any test living systems (i.e., patients or subjects, including control and non-control patients or subjects) and any biological fluid samples can find use with the methods of the present disclosure. Any biological sample which contains or is expected to contain cargo molecules can find use with the methods of the present disclosure. Examples of motoneuron samples include but are not limited to, sciatic or peripheral nerve tissue and samples from the motor cortex of the brain sampling cerebrospinal fluid (CSF). Other samples that find use with the present methods include blood, urine or tissue samples.

In some embodiments, a plurality of samples includes one or more samples. In some embodiments, a plurality of samples includes 1, 2, 3, 4, 5, 6, 7, or 8 or more samples. In some embodiments, a plurality of samples includes 1, 2, or 3 samples. In some embodiments, a plurality of samples includes 1 or 2 samples. In some embodiments, a plurality of samples includes 1 sample.

### CSF Fractionation for Cargo Molecule Analysis

In practicing the method of the invention, in some embodiments, cargo molecules are obtained by the process described below. However, one of skill in the art would understand that other processes for isolating cargo molecules are also contemplated by the methods of the present disclosure and that any cargo molecules isolation methods can be employed with the methods of the present disclosure. Cargo molecules can be obtained from any samples as described herein. Any of the cargo molecules described herein can be quantified. In some embodiments, the cargo molecule detected, analyzed and/or quantitated include alpha-synuclein, chromogranin B, chromogranin A, sAPP, VGF, neuregulin-1, clusterin. proenkephalin-A, galanin, and/or brain-derived neurotrophic factor (BDNF) and any combinations thereof according the methods described herein.

²H₂O labeled CSF is kept frozen −80° C. without any freeze/thaw cycle after collection.

Samples are then thawed and centrifuged at 100,000×g. 80 minutes at 4° C. The pellet contains the cells and the supernatant is carried forward.

The supernatant is then subjected to immuno-depletion of albumin and IgG.

The albumin and IgG immuno-depleted CSF is subjected to reverse-phase chromatography and glycoprotein-fractionation kit.

The fractionated CSF enriched with cargo molecules is subjected to trypsin digestion and LC MS-MS analysis.

Calculations of turnover rates for ²H₂O labeled brain molecules and secretion rates of ²H₂O labeled cargo molecules are performed as described herein as well as in U.S. Publication No. 2014/0274785 and PCT Publication No. WO 2009/143365, both of which are incorporated by reference herein in their entireties.

### Cargo Molecule Analysis

Cargo molecules such as proteins may be partially purified and/or isolated from one or more biological samples, depending on the assay requirements. Any of the cargo molecules described herein can be quantified in the patient samples. In some embodiments, the cargo molecule detected, analyzed and/or quantitated include alpha-synuclein, chromogranin B, chromogranin A, sAPP, VGF, neuregulin-1, clusterin. proenkephalin-A, galanin, brain-derived neurotrophic factor (BDNF) and any combinations thereof according the methods described herein.

In general, cargo molecules can be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, fast performance liquid chromatography (FPLC), chemical extraction, thin layer chromatography, gas chromatography, and chromatofocusing. For example, some proteins may be purified using a standard antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the assay and components of the system. In some instances no purification will be necessary.

In another embodiment, the proteins may be hydrolyzed or otherwise degraded to form smaller molecules. Hydrolysis methods include any method known in the art, including, but not limited to, chemical hydrolysis (such as acid hydrolysis) and biochemical hydrolysis (such as peptidase degradation). Hydrolysis or degradation may be conducted either before or after purification and/or isolation of the proteins. The proteins also may be partially purified, or optionally, isolated, by conventional purification methods including HPLC, FPLC, gas chromatography, gel electrophoresis, and/or any other methods of separating chemical and/or biochemical compounds known to those skilled in the art.

Isotopic enrichment in proteins can be determined by various methods known in the art such as NMR, laser spectroscopy, liquid scintillation counting, Geiger counter, and mass spectrometry. For methods using mass spectrometry, there are several different types of mass spectrometers finding use in the present invention including but not limited to, gas chromatography-mass spectrometry (GC-MS), isotope-ratio mass spectrometry, GC-isotope ratio-combustion-MS, GC-isotope ratio-pyrrolysis-MS, liquid chromatography-MS, electrospray ionization-MS, matrix assisted laser desorption-time of flight-MS, Fourier-transform-ion-cyclotron-resonance-MS, and cycloidal-MS.

Mass spectrometers convert molecules such as proteins into rapidly moving gaseous ions and separate them on the basis of their mass-to-charge ratios. The distributions of isotopes or isotopologues of ions, or ion fragments, may thus be used to measure the isotopic enrichment in a plurality of proteins.

Generally, mass spectrometers include an ionization means and a mass analyzer. A number of different types of mass analyzers are known in the art. These include, but are not limited to, magnetic sector analyzers, electrospray ionization, quadrupoles, ion traps, time of flight mass analyzers, and Fourier transform analyzers.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization.

In addition, two or more mass analyzers may be coupled (MS/MS) first to separate precursor ions, then to separate and measure gas phase fragment ions. These instruments generate an initial series of ionic fragments of a protein and then generate secondary fragments of the initial ions.

Different ionization methods are also known in the art. One key advance has been the development of techniques for ionization of large, non-volatile macromolecules including proteins. Techniques of this type have included electrospray ionization (ESI) and matrix assisted laser desorption. These have allowed MS to be applied in combination with powerful sample separation introduction techniques, such as liquid chromatography and capillary zone electrophoresis.

In addition, mass spectrometers may be coupled to separation means such as gas chromatography (GC), QTOF (quadrupole time-of-flight) mass spectrometry and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer. In the QTOF (quadrupole time-of-flight) mass spectrometry trypsin-digested peptides are analyzed both for protein/peptide identification in data-dependent MS/MS mode and for peptide isotope analysis in MS mode. The QTOF mass spectrometry method is further discussed by Holmes et al. 2015 (Dynamic proteomic: in vivo protein kinetics using metabolic labeling, Methods in Enzymology, Volume 561, pages 216-276) which is hereby incorporated by reference in their entirety.

In general, in order to determine a baseline mass isotopomer frequency distribution for the protein, such a sample is taken before infusion of an isotopically labeled precursor. Such a measurement is one means of establishing in the cell, tissue or organism, the naturally occurring frequency of mass isotopomers of the protein. When a cell, tissue or organism is part of a population of subjects having similar environmental histories, a population isotopomer frequency distribution may be used for such a background measurement. Additionally, such a baseline isotopomer frequency distribution may be estimated, using known average natural abundances of isotopes. For example, in nature, the natural abundance of ¹³C present in organic carbon is 1.11%. Methods of determining such isotopomer frequency distributions are discussed below. Typically, samples of the protein are taken prior to and following administration of an isotopically labeled precursor.

In one embodiment, the relative and absolute mass isotopomer abundances are measured. Measured mass spectral peak heights, or alternatively, the areas under the peaks, may be expressed as ratios toward the parent (zero mass isotope) isotopomer. It is appreciated that any calculation means which provide relative and absolute values for the abundances of isotopomers in a sample may be used in describing such data, for the purposes of the present invention.

In one embodiment, the labeled:unlabeled proportion of cargo molecules such as proteins is calculated. The proportion of labeled and unlabeled molecules of interest (e.g., cargo molecules as described herein) is then calculated. The practitioner first determines measured excess molar ratios for isolated isotopomer species of a molecule. The practitioner then compares measured internal pattern of excess ratios to the theoretical patterns. Such theoretical patterns can be calculated using the binomial or multinomial distribution relationships as described in U.S. Pat. Nos. 5,338,686, 5,910,403, and 6,010,846, which are hereby incorporated by reference in their entirety. The calculations may include Mass Isotopomer Distribution Analysis (MIDA). Variations of Mass Isotopomer Distribution Analysis (MIDA) combinatorial algorithm are discussed in a number of different sources known to one skilled in the art. The method is further discussed by Hellerstein and Neese (1999), as well as Chinkes, et al. (1996), and Kelleher and Masterson (1992), and U.S. patent application Ser. No. 10/279,399, all of which are hereby incorporated by reference in their entirety.

In addition to the above-cited references, calculation software implementing the method is publicly available from Professor Marc Hellerstein, University of California In addition, mass spectrometers, Berkeley.

The comparison of excess molar ratios to the theoretical patterns can be carried out using a table generated for a molecule of interest, or graphically, using determined relationships. From these comparisons, a value, such as the value p, is determined, which describes the probability of mass isotopic enrichment of a subunit in a precursor subunit pool. This enrichment is then used to determine a value, such as the value Aₓ*, which describes the enrichment of newly synthesized proteins for each mass isotopomer, to reveal the isotopomer excess ratio which would be expected to be present, if all isotopomers were newly synthesized.

Fractional abundances are then calculated. Fractional abundances of individual isotopes (for elements) or mass isotopomers (for molecules) are the fraction of the total abundance represented by that particular isotope or mass isotopomer. This is distinguished from relative abundance, wherein the most abundant species is given the value 100 and all other species are normalized relative to 100 and expressed as percent relative abundance. For a mass isotopomer Mₓ,

$$Fractional abundance of {M}_{x} = {A}_{x} = \frac{Abundance {M}_{x}}{{\sum }_{i = 0}^{n} Abundance {M}_{i}} ,$$

where 0 to n is the range of nominal masses relative to the lowest mass (M₀) mass isotopomer in which abundances occur.

$$Δ Fractional abundance ( enrichment or depletion ) = {( {A}_{x} )}_{e} - {( {A}_{x} )}_{b} = {( \frac{Abundance {M}_{x}}{{\sum }_{i = 0}^{n} Abundance {M}_{i}} )}_{e} - {( \frac{Abundance {M}_{x}}{{\sum }_{i = 0}^{n} Abundance {M}_{i}} )}_{b} ,$$

where subscript e refers to enriched and b refers to baseline or natural abundance.

In order to determine the fraction of cargo molecules that were actually newly synthesized during a period of precursor administration, the measured excess molar ratio (EMₓ) is compared to the calculated enrichment value, Aₓ*, which describes the enrichment of newly synthesized cargo molecules (e.g., proteins) for each mass isotopomer, to reveal the isotopomer excess ratio which would be expected to be present, if all isotopomers were newly synthesized.

In one embodiment, molecular flux rates are calculated. The method of determining the synthesis rate of cargo molecules includes calculating the proportion of mass isotopically-labeled cargo molecules in the precursor pool, and using this proportion to calculate an expected frequency of a cargo molecules containing at least one mass isotopically-labeled portion (or the protein being isotopically labeled). This expected frequency is then compared to the actual, experimentally determined isotopomer frequency. From these values, the proportion of cargo molecules which is generated after added isotopically-labeled precursors during a selected incorporation period can be determined Thus, the rate of synthesis during such a time period is also determined. In a system at steady-state concentrations, or when any change in concentrations in the system are measurable or otherwise known during said time period, the rate of disassembly is thereby known as well, using calculations known in the art. A precursor-product relationship is then applied. For the continuous labeling method, the isotopic enrichment is compared to asymptotic (e.g., maximal possible) enrichment and kinetic parameters (e.g., synthesis rates) are calculated from precursor-product equations. The fractional synthesis rate (ks) may be determined by applying the continuous labeling, precursor-product formula:

kₛ=[−ln(1−f)]/t,

where f=fractional synthesis=product enrichment/asymptotic precursor/enrichment and t=time of label administration of contacting in the system studied.

For the pulse-chase ²H₂O (heavy water) labeling method, the rate of decline in isotope enrichment is calculated and the kinetic parameters are calculated using exponential decay equations. Since the heavy water administration only occurred for a short period (e.g. seven days), any isotopic enrichment in cargo molecules present in CSF that persisted after cessation of heavy water intake, is attributed to a certain transport rate of cargo molecules. The first step in quantifying both the transport (e.g., axonal transport) rates of cargo molecules is to calculate the minimum heavy water enrichment that would have resulted in the isotopically-labeled subpopulation of a cargo molecules.

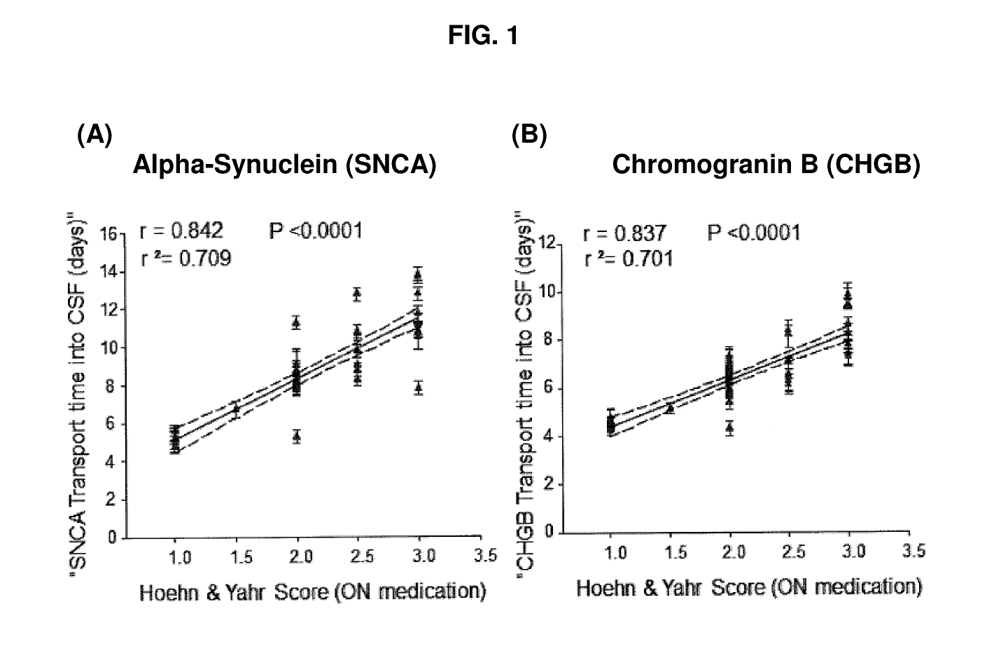

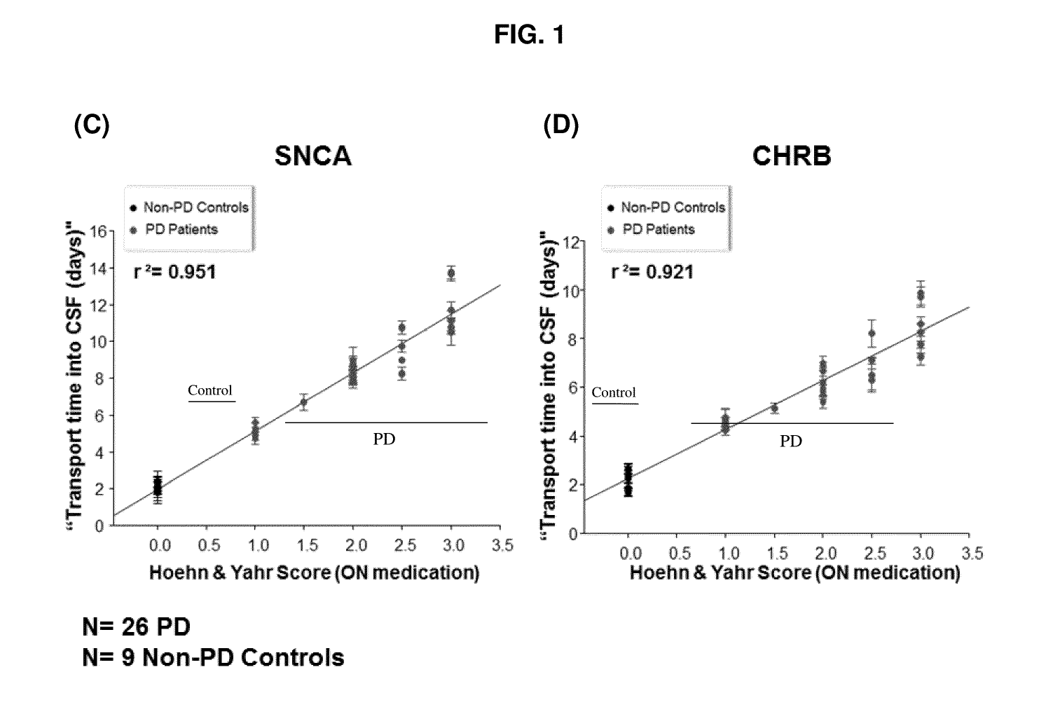

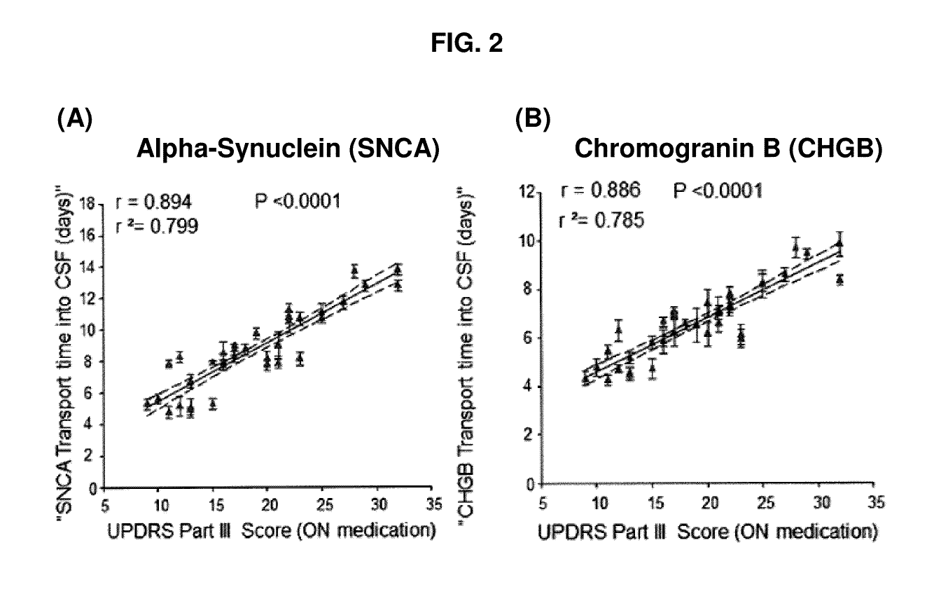

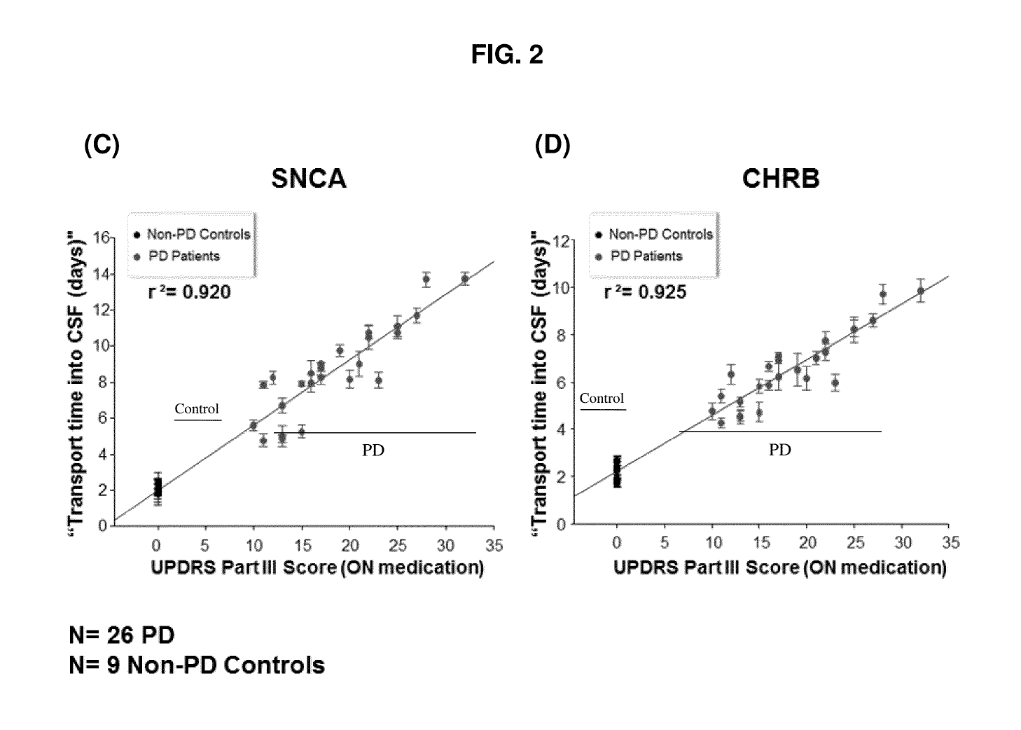

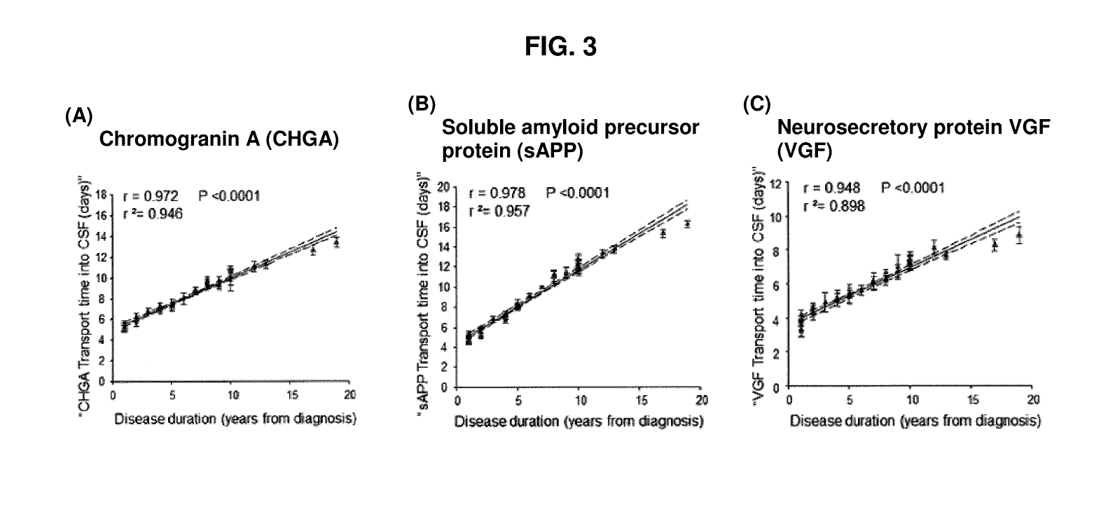

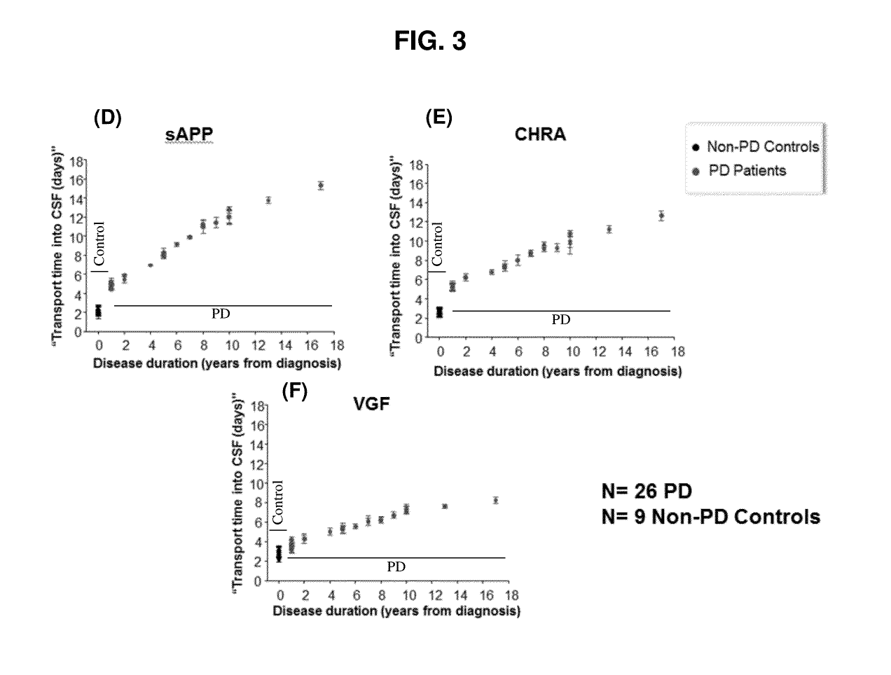

The isotopic enrichment in a newly-synthesized molecule (|EM₀*|) that results from any given ²H-enrichment in body water (p) can be predicted by the use of combinatorial probabilities based on the summed total number of proton incorporation sites (n) in the peptide (Hellerstein & Neese, 1999; Price, Holmes, et al., 2012). For a tryptic peptide from a newly-synthesized protein, the relationship between |EM₀*| and the ²H-enrichment in body water (p) at the time of protein synthesis can be described by a quadratic equation of the form |EM₀*|=A*p²+B*p (FIG. 1). Therefore, given the measured |EM₀| in a peptide, the quadratic equation is used to calculate for the minimum ²H-enrichment in the body water that must have been present at the time the protein was generated, the “born on p.”

$$“ Born on p ” = \frac{- B - \sqrt{{B}^{2} + 4 A \vert EM 0 \vert}}{2 A} \qquad ( 1 )$$

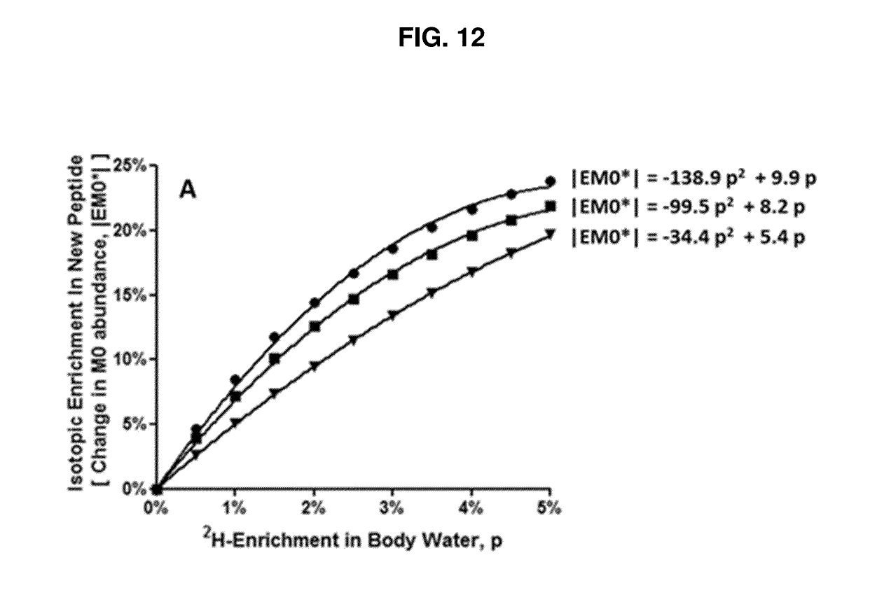

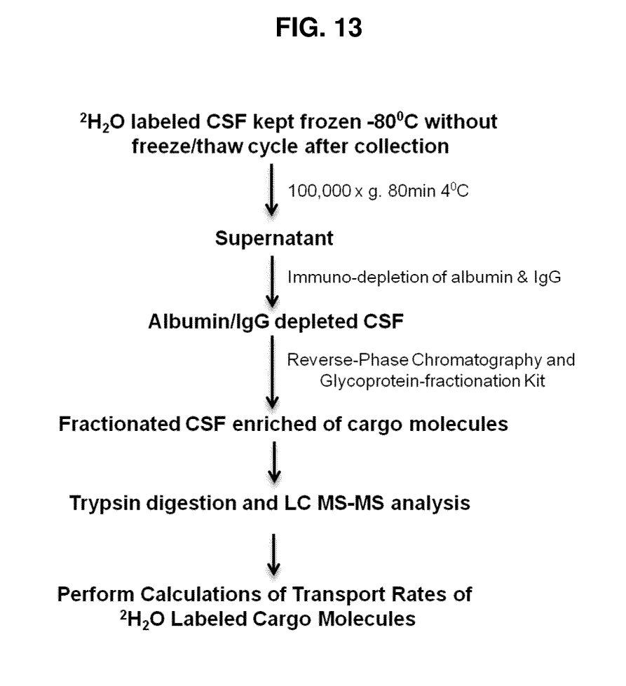

Isotopic enrichment of 3 representative peptides synthesized at various levels of body water enrichment. Relationships can be described by a quadratic fit through the origin (see, FIG. 12).

Next, the molecules “born on p” is related to the subject's history of precursor ²H-enrichment as measured in the subject's body water (²H₂O). Since CSF samples are taken during the washout phase of the study (decline in isotope enrichment), after the subject had stopped taking heavy water, persistent ²H-label enrichment in cargo molecules (i.e., cargo proteins) could represent a time-shifted lag between the synthesis of the protein and the appearance of the protein in the CSF (or other patient sample as described herein). In some cases, the degree of measured labeled cargo molecules in a subject could not have been made on the sampling day (when there was not sufficient heavy water present to achieve that degree of isotopic enrichment measured); those cargo molecules must have been made when there was more heavy water present.

Body water enrichment (²H₂O) measurements from samples taken during the washout phase of the study (decline in isotope enrichment), after the subject had stopped taking heavy water, are modeled as a single phase decay in Graphpad Prism to describe the time-dependent die-away of ²H-enrichment from the subject's body water by a single exponential equation p=P₀e^{-rt}, for t>7 days (after the period of heavy water administration), where P₀ is the extrapolated value for p at Day 0, and r is the best fit parameter corresponding to the subjects' total body water turnover rate (typically 8%/d; Raman, A., D. A. Schoeller, et al. (2004). “Water turnover in 458 American adults 40-79 yr of age.” Am J Physiol Renal Physiol 286(2): F394-401). From this equation describing the timecourse of ²H-enrichment during ²H₂O washout, a “Born-on day” is calculated from the “Born on p” of Equation 1.

$$“ Born on day ” = \frac{- ln ( \frac{“ Born on p ”}{{P}_{0}} )}{r} \qquad ( 2 )$$

Finally, cargo molecule transport rates are calculated based on the difference between the sampling date and the calculated “Born on day” for the cargo molecules. This value represents the average time (in days) between the time of synthesis of the cargo molecules and the appearance of the cargo molecules into CSF (or other patient sample, as described herein).

Usually, the first time point is at least 2-3 hours after administration of precursor (e.g., ²H₂O) has ceased, depending on mode of administration, to ensure that the proportion of mass isotopically labeled subunit (e.g., a labeled cargo molecule) has decayed substantially from its highest level following precursor administration. In one embodiment, the following time points are typically 1-4 hours after the first time point, but this timing will depend upon the replacement rate of the biopolymer pool.

The rate of decay for the cargo molecule is determined from the decay curve for the isotope-labeled subunit. In the present case, where the decay curve is defined by several time points, the decay kinetics can be determined by fitting the curve to an exponential decay curve, and from this, determining a decay constant.

Breakdown rate constants (k_{d}) may be calculated based on an exponential or other kinetic decay curve:

k_{d}=[−ln f]/t.

In one embodiment, the methods of the invention allow mapping of systemic biomarkers associated with amyotrophic lateral sclerosis (ALS) and Parkinson's disease (PD) with or without dementia to specific areas in the brain. Specifically, samples of blood, urine or CSF, for example, can be collected from healthy subject and subjects with amyotrophic lateral sclerosis (ALS) and Parkinson's disease (PD) and the biochemical markers from the samples can be analyzed. In specific embodiments, the location of the brain affect by the motoneuron disease is known. When the location of the brain affected by amyotrophic lateral sclerosis (ALS) and Parkinson's disease (PD) and associated dementia's is known, biochemical markers detected in the blood, urine or CSF that are linked to a motoneuron disease can be further linked (i.e., mapped) to a specific location in the brain.

In certain non-limiting embodiments, the motoneuron disease that is mapped to a specific location of the brain is Parkinson's disease (PD), Parkinson's disease dementia (PDD), and/or amyotrophic lateral sclerosis (ALS).

In certain non-limiting embodiments, cargo molecules, cargo molecule rate of transport (and their correlated motoneuron diseases) can be mapped to the cerebrum, cerebellum, limbic system, brain stem, the frontal lobe, the parietal lobe, the occipital lobe, the temporal lobe, the thalamus, the hypothalamus, the amygdale, the hippocampus, the midbrain, the pons, or the medulla.

### Pharmaceutical Compositions for Treatment

As outlined herein, there are a variety of pharmaceutical compositions that can be used to treat motoneuron diseases, particularly Parkinson's disease (PD) or amyotrophic lateral sclerosis (ALS) and can be included in the treatment methods described throughout this disclosure. In one embodiment, the pharmaceutical composition comprises an MTMA agent and a pharmaceutical carrier, as outlined herein. In this embodiment, noscapine finds particular use.

Treatment can include any pharmaceutical formulations as described herein or used for treatment of PD. In some embodiments, the treatment includes drugs currently in clinical trials for ALS and/or PD. In some embodiments, the treatment comprises administration of rasagiline (Azilect). In some embodiments, the treatment comprises administration of cholinesterase inhibitors (e.g., donepezil). In one embodiment, pharmacokinetic treatment profiles for Parkinson's disease (PD) are determined for rasagiline (Azilect). In one embodiment, pharmacokinetic treatment profiles for Parkinson's disease (PD) are determined for cholinesterase inhibitors (e.g., donepezil).

In many embodiments, the pharmaceutical compositions comprise two different drug agents. Any combination of any two types of neuroprotective agents outlined herein is possible. In some cases, three neuroprotective agents can be combined for treatment.

In one embodiment, the pharmaceutical composition comprises two different MTMAs; for example, noscapine and a cannabinoid, including an endocannabinoid, find use in this embodiment.

In alternative embodiments, the pharmaceutical compositions comprise an MTMA and a neuroprotective agent that is not an MTMA.

In one embodiment, the neuroprotective agent is a voltage gated ion channel antagonist, including voltage gated sodium and calcium channel antagonists. Thus, compositions comprising at least one MTMA and a channel antagonist find particular use.

In many embodiments, the pharmaceutical compositions comprise an MTMA and an NMDA receptor antagonist. Compositions comprising noscapine and dizolcipine find particular use in some embodiments. In alternative embodiments, the NMDA receptor antagonist is Memamtine.

In many embodiments, the pharmaceutical compositions comprise an MTMA and a peroxisome proliferator-activated receptor gamma (PPARγ) agonist. Compositions comprising noscapine and pioglitazone (Actos®) find particular use in some embodiments. In alternative embodiments, the PPARγ agonist can be Rosiglitazone (Avandia®), L-796449, RS5444, or GI262570 among others.

In many embodiments, the pharmaceutical composition comprises MTMA and an anti-inflammatory agent, such as Celastrol, Nimesulide or Ibuprofen.

In many embodiments, the pharmaceutical compositions comprise an MTMA and an antioxidant, particularly iNOS antioxidants. Compositions comprising noscapine and L-NMMA (Tilarginine) find particular use in some embodiments. In alternative embodiments, the antioxidant can be chosen from Ceftriaxone, Celastrol, CoQ10, Vitamin E, or AEOL 10150 among others.

In many embodiments, the pharmaceutical compositions comprise an MTMA and a free radical trapper/scavenger. Compositions comprising noscapine and manganoporphyrin antioxidant among others find use in some embodiments.

In many embodiments, the pharmaceutical compositions comprise an MTMA and a metal ion chelator, particularly copper(II) and zinc(II) chelators. Compositions comprising noscapine and a metal ion chelator such as 8-hydroxyquinoline; acetohydroxamic acid; or N,N-dimethyl-2,3-dihydroxybenzamide (DMB), among others, find use in some embodiments.

In many embodiments, the pharmaceutical compositions comprise an MTMA and a low-voltage sensitive calcium channel (L-VSCCs) antagonist. Compositions comprising noscapine and Nimodipine find particular use in some embodiments.

In many embodiments, the pharmaceutical compositions comprise an MTMA and a noncompetitive α-amino-3-hydroxy-5-methylisoxazole-4-propionic acid (AMPA)/kainate receptor antagonist. Compositions comprising noscapine and GYKI 52466 find particular use in some embodiments.

In many embodiments, the pharmaceutical compositions comprise an MTMA and a selective or nonselective glutamate receptor antagonist. Compositions comprising noscapine and the nonselective glutamate receptor antagonist Sosei 51 (NC-1200/MVL-6976) find particular use in some embodiments. In alternative embodiments, the selective or nonselective glutamate receptor antagonist can be chosen from NBQX, Nimesuldine, Riluzole (Rilutek), Talampanel, Ceftriaxone, or Naaladase inhibitor. In other embodiments, the glutamate receptor antagonist may be a glial modulator such as ONO-2506.

In many embodiments, the pharmaceutical compositions comprise an MTMA and an Anandamide (AEA) transport, hydrolysis or reuptake inhibitor. Compositions comprising noscapine and N-(4-hydroxyphenyl)-arachidonamide (AM404) find particular use in some embodiments. In alternative embodiments, the AEA transport, hydrolysis or reuptake inhibitor may be N-(5Z, 8Z, 11Z, 14Z eicosatetraenyl)-4-hydroxybenzamide (AM1172) or a fatty acid amidohydrolase FAAH inhibitor, such as URB597. In addition, compositions comprising two MTMAs and an AEA reuptake inhibitor are also useful, such as noscapine, AEA and AM404.

In many embodiments, the pharmaceutical compositions comprise an MTMA and a neurotrophic factor. Compositions comprising noscapine and IGF-1 or IGF-1-AAV find use in some embodiments.

In many embodiments, the pharmaceutical compositions comprise an MTMA and an apoptosis inhibitor. Compositions comprising noscapine and Minocycline, TCH346 or Tamoxifen may find use in some embodiments.

In some embodiments, two MTMAs are used as well as an additional neuroprotective agent.

By “motoneuron related disorder” or “motoneuron disease” or “condition” herein is meant a disorder that can be ameliorated by the administration of a pharmaceutical composition comprising two neuroprotective agents, typically comprising at least one MTMA, although one and more than two neuroprotective agents are also contemplated. In a particularly useful embodiment, the microtubule target modulating agent is used to treat amyotrophic lateral sclerosis (ALS) or Parkinson's disease (PD) with or without dementia.

In many embodiments, a therapeutically effective dose of neuroprotective agents is administered to a patient in need of treatment. By “therapeutically effective dose” herein is meant a dose that produces the effects for which it is administered. The exact dose will depend on the purpose of the treatment, and will be ascertainable by one skilled in the art using known techniques. In a particularly useful embodiment, dosages of about 5 μg/kg are used, administered either intravenously or subcutaneously. As is known in the art, adjustments for agent degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In a particularly useful embodiment the patient is a mammal, and in an especially useful embodiment the patient is human.

As the compositions of the invention are typically combinations of at least two neuroprotective agents, the compositions can be administered together in a single dosage form (e.g., oral formulations that combine the two drugs) or singly, in any of the dosage forms outlined below, simultaneously or sequentially. For example, one drug can be administered orally and another intraperitoneally, either together or sequentially. In addition, when dosed separately, the dosages may be at different times or frequencies. Alternatively, at least two drugs may be administered separately but in the same dosage form, e.g., by oral administration.

Initial dosages suitable for administration to humans may be determined from in vitro assays or animal models. For example, an initial dosage may be formulated to achieve a serum concentration that includes the IC₅₀ of the particular metabolically active agent of the compound(s) being administered, as measured in an in vitro assay. Alternatively, an initial dosage for humans may be based upon dosages found to be effective in animal models of ALS, such as the SOD mouse. As one example, the initial dosage for each component of the pharmaceutical compositions outlined herein may be in the range of about 0.01 mg/kg/day to about 200 mg/kg/day, or about 0.1 mg/kg/day to about 100 mg/kg/day, or about 1 mg/kg/day to about 50 mg/kg/day, or about 10 mg/kg/day to about 50 mg/kg/day, can also be used. The dosages, however, may be varied depending upon the requirements of the patient, the severity of the condition being treated, and the compound(s) being employed. The size of the dose also will be determined by the existence, nature, and extent of any adverse side-effects that accompany the administration of a particular compound(s) in a particular patient. Determination of the proper dosage for a particular situation is within the skill of the practitioner. Generally, treatment is initiated with smaller dosages which are less than the optimum dose of the compound(s). Thereafter, the dosage is increased by small increments until the optimum effect under circumstances is reached. For convenience, the total daily dosage may be divided and administered in portions during the day, if desired.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Formulations suitable for oral administration can consist of (a) liquid solutions, such as an effective amount of the compound(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition.

The active compound or pharmaceutically acceptable salt thereof can be administered as a component of an elixir, suspension, syrup, wafer, chewing gum or the like. Syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action.

As used herein, the term “pharmaceutically acceptable salt(s)” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counter-ion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The compound(s) of choice, alone or in combination with other suitable components, may be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged compound(s) with a suppository base. Suitable suppository bases include natural or synthetic triglycerides or paraffin hydrocarbons. In addition, it is also possible to use gelatin rectal capsules which consist of a combination of the compound(s) of choice with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, subcutaneous administration and intravenous administration are particularly useful methods of administration. A specific example of a suitable solution formulation may comprise from about 0.1-100 mg/ml compound(s) and about 1000 mg/ml propylene glycol in water. Another specific example of a suitable solution formulation may comprise from about 0.1 or about 0.2 to about 100 mg/ml compound(s) and from about 800-1000 mg/ml polyethylene glycol 400 (PEG 400) in water.

A specific example of a suitable suspension formulation may include from about 0.2-30 mg/ml compound(s) and one or more excipients selected from the group consisting of: about 200 mg/ml ethanol, about 1000 mg/ml vegetable oil (e.g., corn oil), about 600-1000 mg/ml fruit juice (e.g., grape juice), about 400-800 mg/ml milk, about 0.1 mg/ml carboxymethylcellulose (or microcrystalline cellulose), about 0.5 mg/ml benzyl alcohol (or a combination of benzyl alcohol and benzalkonium chloride) and about 40-50 mM buffer, pH 7 (e.g., phosphate buffer, acetate buffer or citrate buffer or, alternatively 5% dextrose may be used in place of the buffer) in water.

A specific example of a suitable liposome suspension formulation may comprise from about 0.5-30 mg/ml compound(s), about 100-200 mg/ml lecithin (or other phospholipid or mixture of phospholipids) and optionally about 5 mg/ml cholesterol in water. For subcutaneous administration of a compound(s), a liposome suspension formulation including 5 mg/ml compound(s) in water with 100 mg/ml lecithin and 5 mg/ml compound(s) in water with 100 mg/ml lecithin and 5 mg/ml cholesterol provides good results.

The formulations of compound(s) can be presented in unit-dose or multi-dose sealed containers, such as ampoules and vials. Injection solutions and suspensions can be prepared from sterile powders, granules, and tablets of the kind previously described.

The pharmaceutical preparation is particularly useful in unit dosage form. In such form the preparation is subdivided into unit doses containing appropriate quantities of the compound(s). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents, discussed in more detail, below.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation (Mountain View, Calif.) and Gilford Pharmaceuticals (Baltimore, Md.). Liposomal suspensions also may be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidylcholine, arachadoyl phosphatidylcholine, and cholesterol) in an inorganic solvent that is then evaporated, leaving behind a thin film of dried lipid on the surface of the container. Aqueous solutions of the active compound or its derivatives are then introduced into the container. The container is then swirled by hand to free lipid material from the sides of the container and to disperse lipid aggregates, thereby forming the liposomal suspension.

### EXAMPLES

### Example 1

### Human Study Design and ²H₂O Labeling

The study group consisted of symptomatic PD patients, all on standard anti-PD medications, and 6 non-PD volunteers. PD diagnosis was based on clinical evaluation and UK Parkinson's Disease Society Brain Bank Clinical Diagnostic Criteria (Hughes A J, Daniel S E, Kilford L, Lees A J., J Neurol Neurosurg Psychiatry, 55(3):181-184 (1992)). Unified PD Rating Scale, PART III, and Hoehn and Yahr scores were obtained for each subject. None of the patients had any atypical features or evidence of more widespread neurological disease. The 6 non-PD controls included 3 healthy volunteers and 3 individuals with well-treated HIV infection; the latter were on combination antiretroviral therapy with long-term plasma viral suppression, had normal CSF white blood cell counts, and CSF/plasma albumin ratios indicated absence of local inflammation or abnormal blood-brain barrier permeability. Eligible subjects were instructed to drink 50 ml of 70% ²H₂O (Isotec-Sigma) 3 times per day, leaving at least 3 hours between doses, for a period of 7 consecutive days. Compliance to the experimental protocol was monitored by the clinical staff through collection of blood plasma samples for body water enrichment. 4 LPs were conducted in 6 controls and 1 PD patient (days 2-15 and 21-43), and a single LP was conducted in the other 11 PD subjects (days 15, 21, 22, or 23), after starting ²H₂O administration (7 days labeling protocol).

Processing of cargo proteins for GC/MS analysis. Purified cargo proteins were hydrolyzed by treatment with 6N HCl for 16 hours at 110° C. Protein derived amino acids were derivatized to pentafluorobenzyl derivatives, and ²H incorporation in alanine released from total vesicular proteins was measured by GC/MS, as described elsewhere (Busch R, et al., Biochim Biophys Acta., 1760(5):730-44 (2006)). ²H enrichment was calculated as the percent increase over the natural abundance of alanine derivative present as the (M+1) mass isotopomer (EM1) (Busch R, et al., Biochim Biophys Acta., 1760(5):730-44 (2006)). Calculations. The fraction of newly synthesized alanine in each sample was calculated as the ratio of the measured EM1 value to the maximal value expected at the measured body water enrichment, which was calculated by mass isotopomer distribution analysis, as described in detail elsewhere (Busch R, et al., Biochim Biophys Acta., 1760(5):730-44 (2006)). This value was taken to represent fractional protein synthesis.

Statistics. The statistical significance of CSF secretion rates of selected cargo molecules was assessed by 1-way ANOVA with Tukey post-hoc testing. A P value less than 0.05 was considered statistically significant. Software for statistics included SigmaStat3.0 and Microsoft Excel 2003.

See, Fanara et. al., JCI Clin Invest. doi:10.1172/JCI64575; incorporated herein by reference in its entirety.

We claim:

1. A method of diagnosing or predicting the presence of Parkinson's disease (PD) in a patient comprising:
a) examining a plurality of patient samples obtained over one or more timepoints from said patient, wherein said samples comprise isotope-labeled cargo molecules;
b) quantifying the rate of transport of one or more cargo molecules in said samples;
c) comparing the rate of transport of said one or more cargo molecules in said patient samples to the rate of transport of said one or more cargo molecules in control subject samples,
wherein detecting a decrease in the rate of transport of said one or more cargo molecules in said patient samples as compared to the rate of transport of said one or more cargo molecules in said control subject samples is indicative of Parkinson's disease.

2. The method of claim 1, wherein said method comprises a step of administering an isotope-labeled substrate to said patient for a period of time sufficient for said isotope-label to be detectable in said patient sample, wherein said administration is prior to step a).

3. The method of claim 1, wherein said one or more cargo molecules is selected from the group consisting of alpha-synuclein, chromogranin B, chromogranin A, sAPP, and VGF, and combinations thereof.

4. The method of claim 1, wherein a decrease in the rate of transport of said one or more cargo molecules is selected from the group consisting of chromogranin A, sAPP, and VGF is indicative of longer disease duration.

5. The method of claim 4, wherein chromogranin A, sAPP, or VGF do not correlate with disease severity.

6. The method of claim 1, wherein a decrease in the rate of transport of said one or more cargo molecules selected from the group consisting of alpha-synuclein and chromogranin B is indicative of degenerating neurons linked to motor symptoms of PD.

7. The method of claim 1, wherein the decrease in the rate of transport of said one or more cargo molecules selected from the group consisting of alpha-synuclein and chromogranin B correlates with clinical disease stage, wherein a greater decrease in rate of transport is indicative of a more advanced clinical disease stage and/or disease severity.

8. The method of claim 1, wherein said one or more cargo proteins is not neuregulin-1 or clusterin.

9. The method of claim 1, wherein said sample is selected from the group consisting of CSF, blood, urine, and tissue.

10. The method of claim 9, wherein said sample is CSF.

11. A method of diagnosing or predicting the presence of Parkinson's disease dementia (PDD) in a patient previously diagnosed with Parkinson's disease, the method comprising:
a) examining a plurality of patient samples obtained over one or more timepoints from said patient, wherein said samples comprise isotope-labeled cargo molecules;
b) quantifying the rate of transport of one or more cargo molecules in said patient samples;
c) comparing the rate of transport of said one or more cargo molecules in said patient samples to the rate of transport of said one or more cargo molecules in control subject samples, and
d) detecting a decrease in the rate of transport of said one or more cargo molecules in said patient samples as compared to the rate of transport of one or more cargo molecules in said control subject samples,
wherein the decrease in the rate of transport of said one or more cargo molecules into samples from a subject with PDD as compared to the rate of transport of said one or more cargo molecules into samples from a control subjects is greatly different for a subject with PD as compared to a control subject.

12. The method of claim 11, wherein said method comprises a step of administering an isotope-labeled substrate to said patient for a period of time sufficient for said isotope-label to be detectable in said patient sample, wherein said administration is prior to step a).

13. The method of claim 11, wherein a greater decrease in the rate of transport in said patient samples as compared to said control subject samples results in a greater increase in the probability of developing dementia.

14. The method of claim 11, wherein said one or more cargo molecules is selected from the group consisting of proenkephalin-A and galanin.

15. The method of claim 11, wherein said sample is selected from the group consisting of CSF, blood, urine, and tissue.

16. The method of claim 15, wherein said sample is CSF.

17. A method of differentiating between Parkinson's disease (PD) and amyotrophic lateral sclerosis (ALS) in a patient comprising:
a) examining a plurality of patient samples obtained over one or more timepoints from said patient, wherein said samples comprise isotope-labeled cargo molecules;
b) quantifying the rate of transport of one or more cargo molecules into said samples or in said samples;
c) comparing the rate of transport of said one or more cargo molecules in said patient samples to the rate of transport of said one or more cargo molecules in control subject samples, and
d) detecting a difference in the rate of transport of at least two or more cargo molecules in said patient samples as compared to the rate of transport of said one or more cargo molecules in said control subject samples,
wherein (i) a decrease in the rate of transport of alpha-synuclein in said patient samples as compared to the rate of transport of alpha-synuclein in said control subject samples and (ii) no change in the rate of transport of neuregulin-1 in said patient samples as compared to the rate of transport of neuregulin-1 in said control subject samples is indicative of PD, and
wherein (i) a decrease in the rate of transport of neuregulin-1 in said patient samples as compared to the rate of transport of neuregulin-1 in said control subject samples and (ii) no change in the rate of transport of alpha-synuclein in said patient samples as compared to the rate of transport of alpha-synuclein in said control subject samples, is indicative of ALS.

18. The method of claim 17, wherein said method comprises a step of administering an isotope-labeled substrate to said patient for a period of time sufficient for said isotope-label to be detectable in said patient samples, wherein said administration is prior to step a).

19. The method of claim 17, wherein said sample is selected from the group consisting of CSF, blood, urine, and tissue.

20. The method of claim 19, wherein said sample is CSF.

21. A method of monitoring a treatment regimen in Parkinson's disease (PD) in a patient being treated for PD with a drug comprising:
a) examining a plurality of patient samples obtained over one or more timepoints from said patient, wherein said samples comprise isotope-labeled cargo molecules;
b) quantifying the rate of transport of one or more cargo molecules in said samples;
c) comparing the rate of transport of said one or more cargo molecules in said patient samples to the rate of transport of said one or more cargo molecules in PD patient samples,
wherein detecting a reduction in the decrease in the rate of transport of said one or more cargo molecules in said patient samples as compared to the rate of transport of one or more cargo molecules in said PD subject samples is indicative of the therapeutic efficacy of the treatment,
wherein said PD subject samples are from (i) a subject not being treated or (ii) from the same patient prior to beginning treatment.

22. The method of claim 21, wherein said method comprises a step of administering an isotope-labeled substrate to said patient for a period of time sufficient for said isotope-label to be detectable in said patient samples, wherein said administration is prior to step a).

23. The method of claim 21, wherein the treatment comprises administration of a drug selected from the group consisting of Azilect (rasagiline) and cholinesterase inhibitors (e.g. donepezil).

24. The method of claim 21, wherein the one or more cargo molecules is brain-derived neurotrophic factor (BDNF).

25. The method of claim 21, wherein said sample is selected from the group consisting of CSF, blood, urine, and tissue.

26. The method of claim 25, wherein said sample is CSF.
